(19) United States
(12) Patent Application Publication
(10) Pub. No.: US 2026/0090547 A1
(43) Pub. Date: Apr. 2, 2026

(54) BIO-INSECTICIDES FROM WASTE BIOMASS

(71) Applicants: Tejas Goculdas, Newark, DE (US); Sunitha Sadula, Newark, DE (US); Dionisios G. Vachos, Newark, DE (US)

(72) Inventors: Tejas Goculdas, Newark, DE (US); Sunitha Sadula, Newark, DE (US); Dionisios G. Vachos, Newark, DE (US)

(73) Assignee: University of Delaware, Newark, DE (US)

(21) Appl. No.: 19/414,147

(22) Filed: Dec. 9, 2025

Related U.S. Application Data
(63) Continuation-in-part of application No. PCT/US2024/037794, filed on Jul. 12, 2024.
(60) Provisional application No. 63/513,413, filed on Jul. 13, 2023.
(60) Provisional application No. 63/729,654, filed on Dec. 9, 2024.

(51) Int. Cl. A01N 47/12; A01P 7/04; C07D 307/14; C07D 307/16

(52) U.S. Cl. CPC A01N 47/12; A01P 7/04; C07D 307/14; C07D 307/16

(57) ABSTRACT

Disclosed herein are bio-based insecticide active ingredients with carbamate functional group for more sustainable pest management practices. Processes for producing these bio-based insecticide compositions are also disclosed.

### CROSS-REFERENCE TO RELATED APPLICATIONS

This application is a continuation-in-part of International Application No. PCT/US2024/037794, filed on Jul. 12, 2024, which claims the benefit of priority of U.S. Provisional Application No. 63/513,143 filed on Jul. 12, 2023. This application also claims the benefit of priority of U.S. Provisional Application No. 63/729,654 filed on Dec. 9, 2024. The contents of all the priority applications are hereby incorporated by reference in their entireties.

### FIELD

The present disclosure relates to bio-based insecticide compositions for more sustainable pest management practices and methods for producing these bio-based insecticide compositions.

### BACKGROUND

To meet rising population trends, worldwide crop production is projected to increase by 70% by 2050. The current Global Insecticide Market is 18.47 billion USD and, as a consequence of rising population trends, the demand for agricultural insecticides will increase. The carbamate insecticides market size is forecasted to reach a value of $341.6 million by the end of 2027, growing at a compound annual growth rate of 4.5%. This market trend poses a significant environmental threat as commercial carbamate insecticides are synthesized from petroleum derived aromatics and produced from fossil fuel-derived phenols, such as cresol (V. Gagic et al., Proc. Natl. Acad. Sci. U.S.A., 2021, 118). Commonly used insecticides such as fenitrothion, Amitraz and chlorpropham are derived from fossil fuels and are commercially priced at $70 to $240 per gallon. Synthesis of these insecticides involve the use of strong acids (e.g., nitric and sulfuric acids) that ultimately play into the production of toxic waste streams resulting in ecological damage (A. S. Nile et al., Environ. Sci. Pollut. Res., 2019, 26, 21127-21139).

Bio-based insecticides are a more sustainable and often safer alternative for pest management in agricultural production. Unfortunately, progress in this field is rather limited. Bio-oils obtained from the pyrolysis of biomass and other mild fungicides derived from reductive catalytic fractionation of lignin have only been explored. Additionally, the insecticidal activity of these compounds is several orders of magnitude lower than that of commercial insecticides.

Thus, to address the foregoing issues, we disclose herein bio-based insecticide compositions for more sustainable pest management practices and methods for producing these bio-based insecticide active ingredients.

### SUMMARY

Disclosed herein is a first process for preparing a bio-based carbamate functionalized insecticide that includes (i.e., comprises) at least one or more of performing a reductive amination reaction on an aldehyde to produce an amine product; subjecting the amine product to a carbonylation reaction to produce the bio-based carbamate functionalized insecticide, and/or optionally performing a ring hydrogenation reaction on the bio-based carbamate functionalized insecticide.

Disclosed herein is a second process for preparing a bio-based carbamate functionalized insecticide that includes at least one or more of performing a reductive amination reaction on an aldehyde to produce an amine product; subjecting the amine product to an aminolysis reaction to produce the bio-based carbamate functionalized insecticide, optionally performing a ring hydrogenation reaction on the produced carbamate functionalized insecticide, optionally performing an etherification reaction on the produced carbamate functionalized insecticide to produce an ether-containing bio-based carbamate functionalized insecticide, and/or optionally performing a ring hydrogenation on the ether-containing bio-based carbamate functionalized insecticide.

Disclosed herein is a third process for preparing a bio-based carbamate functionalized insecticide that includes at least one or more of performing an azidation reaction on a methyl-ester containing an alkyl halogen group to produce an azide intermediate, subjecting the azide intermediate to an azide reduction reaction to produce an amine intermediate, performing an aminolysis reaction on the amine intermediate to produce a methyl ester carbamate intermediate, subjecting the methyl ester carbamate intermediate to a hydrolysis reaction to produce the bio-based carbamate functionalized insecticide, and/or optionally performing a ring hydrogenation on the bio-based carbamate functionalized insecticide.

Disclosed herein are also compositions including at least one bio-based carbamate functionalized insecticide prepared by the methods disclosed herein.

### BRIEF DESCRIPTION OF THE DRAWINGS

Other features and advantages of the compositions, devices and methods disclosed herein will be apparent to those skilled in the art reading the following detailed description in conjugation with the exemplary embodiments illustrated in the drawings, wherein:

FIG. 1 depicts a reaction network for an exemplary reductive amination reaction with vanillin.

FIG. 2 depicts a catalyst screening and optimizations for an exemplary reductive amination reaction with vanillin. (A) catalyst screening conditions: 7.5 mL liquid ammonia, 0.4 g vanillin, 0.05 g catalyst, 100° C., 60 minutes, 40 bar H₂, 800 rpm. (B) reaction conditions: 7.5 mL liquid ammonia, 0.4 g vanillin, 0.05 g catalyst, 60 minutes, 40 bar H₂, 800 rpm. (C) reaction conditions: 7.5 mL liquid ammonia, 0.4 g vanillin, 0.05 g catalyst, 60 minutes, 80° C., 800 rpm. (D) reaction conditions: 7.5 mL liquid ammonia, 0.4 g vanillin, 0.05 g catalyst, 80° C., 40 bar H₂, 800 rpm.

FIG. 3 depicts an optimization of an exemplary reductive amination reaction of furfural to furfurylamine.

FIG. 4 depicts an optimization of an exemplary reductive amination from furfural to furfurylamine. (A) reaction conditions: 7.5 mL liquid ammonia, 0.25 g furfural, 0.05 g Rh/Al₂O₃, 800 rpm stirring, 60 minutes, 80° C. (B) reaction conditions: 7.5 mL liquid ammonia, 0.25 g furfural, 0.05 g Rh/Al₂O₃, 800 rpm stirring, 60 minutes, 40 bar H₂. (C) reaction conditions: 7.5 mL liquid ammonia, 0.25 g furfural, 0.05 g Rh/Al₂O₃, 800 rpm stirring, 40 bar H₂, 100° C.

FIG. 5 depicts recycle runs for an exemplary reductive amination reaction of furfural to furfurylamine. Reaction conditions: 100° C., 7.5 mL liquid ammonia, 0.25 g furfural, 0.05 g Rh/Al₂O₃, 800 rpm stirring, 60 minutes, 40 bar H₂.

FIG. 6 depicts a reaction mechanism for an exemplary carbonylation reaction of furfurylamine. The reaction mechanism focuses on the coordination of a triflate catalyst to a carbonyl group.

FIG. 7 depicts a reaction mechanism for an exemplary carbonylation reaction of furfurylamine. The reaction mechanism provides possible byproducts of the exemplary carbonylation reaction.

FIG. 8 depicts a catalyst screening for an exemplary carbonylation reaction of furfurylamine. Reaction conditions: 80° C., 10 hours, 30 mmol DMC, 3 mmol furfurylamine, 0.15 mmol catalyst, and 800 rpm stirring. Methyl (2-furylmethyl) carbamate (FC), Tris(furan-2-ylmethyl)amine (TA), N-methyl furfurylamine (MFA), and 1-(2-furyl)-N-(2-furylmethyl) methanimine (DA).

FIG. 9 depicts an optimization of the reactant:solvent molar ratio for an exemplary carbonylation reaction of furfurylamine. Reaction conditions: 80° C., 10 hours, 3 mmol furfurylamine, 0.3 mmol La(OTf)₃ and 800 rpm stirring.

FIG. 10 depicts an optimization of catalyst loading for an exemplary carbonylation reaction of furfurylamine. Reaction conditions: 80° C., 10 hours, 30 mmol DMC, 3 mmol furfurylamine and 800 rpm stirring.

FIG. 11 depicts an optimization of reaction time for an exemplary carbonylation reaction of furfurylamine. Reaction conditions: 80° C., 30 mmol DMC, 3 mmol furfurylamine, 0.18 mmol La(OTf)₃ and 800 rpm stirring.

FIG. 12 depicts a catalyst screening for an exemplary vanillylamine carbonylation reaction. Reaction conditions: 80° C., 10 hours, 30 mmol DMC, 3 mmol vanillylamine, 0.3 mmol La(OTf)₃ and 800 rpm stirring.

FIG. 13 depicts the mortality of adult Alphitobius diaperinus exposed in vial bioassays to carbofuran, methyl (2-furylmethyl) carbamate, or methyl (4-hydroxy-3-methoxybenzyl) carbamate. Lines depict the median lethal concentrations (LC₅₀), and shading depicts the 95% Cl, estimated with probit analysis.

FIG. 14 depicts ecotoxicity simulations for commercial carbamate class insecticides compared with exemplary carbamate compounds prepared by the methods disclosed herein. The LC₅₀ data was generated for plankton. Methyl (2-furylmethyl) carbamate (FC), methyl (4-hydroxy-3-methoxybenzyl) carbamate (VC).

FIG. 15 depicts ecotoxicity simulations for commercial carbamate class insecticides compared with exemplary carbamate compounds prepared by the methods disclosed herein. The LC₅₀ data was generated for fish. Methyl (2-furylmethyl) carbamate (FC), methyl (4-hydroxy-3-methoxybenzyl) carbamate (VC).

FIG. 16 depicts ecotoxicity simulations for commercial carbamate class insecticides compared with exemplary carbamate compounds prepared by the methods disclosed herein. The LD₅₀ data was generated for rats. Methyl (2-furylmethyl) carbamate (FC), methyl (4-hydroxy-3-methoxybenzyl) carbamate (VC).

FIG. 17 depicts toxicity simulations for known biopesticide active ingredients compared with exemplary carbamate compounds prepared by the methods disclosed herein. LC₅₀ data is for fish. Methyl (2-furylmethyl) carbamate (FC), methyl (4-hydroxy-3-methoxybenzyl) carbamate (VC).

FIG. 18 depicts toxicity simulations for known biopesticide active ingredients compared with exemplary carbamate compounds prepared by the methods disclosed herein. LD₅₀ data is for rats. Methyl (2-furylmethyl) carbamate (FC), methyl (4-hydroxy-3-methoxybenzyl) carbamate (VC).

FIG. 19 depicts toxicity simulations for known biopesticide active ingredients compared with exemplary carbamate compounds prepared by the methods disclosed herein. LC₅₀ data is for plankton. Methyl (2-furylmethyl) carbamate (FC), methyl (4-hydroxy-3-methoxybenzyl) carbamate (VC).

FIG. 20 depicts possible environmental fates for end-life products for commercial and biobased insecticides prepared by the methods disclosed herein. (a) Carbofuran. (b) Bio-based insecticides prepared by the methods disclosed herein.

FIG. 21 depicts an exemplary synthetic pathway for making bio-based carbamate functionalized insecticide that involves a reductive amination, a carbonylation and an optional hydrogenation.

FIG. 22 depicts an exemplary synthetic pathway for making bio-based carbamate functionalized insecticide that involves a reductive amination, an aminolysis, an optional an etherification and two optional hydrogenations.

FIG. 23 depicts an exemplary synthetic pathway for making bio-based carbamate functionalized insecticide that involves an azidation, an azide reduction, an aminolysis, a hydrolysis and an optional hydrogenation.

FIG. 24 depicts the results of an exemplary reductive amination (a) and carbonylation (b).

FIG. 25 depicts a reductive amination reaction pathway for 5-hydroxymethyl furfural.

FIG. 26 depicts the results of reductive amination reaction for 5-hydroxymethyl furfural. Reaction conditions: 0.4 g HMF, 0.05 g Rh/Al₂O₃, 10 mL liq. ammonia, 80° C., 50 bar H₂, 60 minutes.

FIG. 27 depicts a possible mechanism for the aminolysis of a chloroformate. R1=Furfuryl alcohol, R2=Methyl group.

FIG. 28 depicts a mechanism for a Williamson ether synthesis that converts HMFC to HMFC OMe. Reaction Conditions: 2 mmol HMFC, 1.1 molar equivalents KtBuO, 1.1 molar equivalents CH₃—I, 3 hours and room temperature.

FIG. 29 depicts a synthetic pathway for converting an amine hydrochloride salt to a carbamate and carboxylic acid substituent.

FIG. 30 depicts lethal concentrations of exemplary bio-based carbamate functionalized insecticides and an industrial reference carbofuran, evaluated against the lesser mealworm beetle, compared against their toxicity toward the fathead minnow.

FIG. 31 depicts observed trends from ecological screening and biological assays.

### DETAILED DESCRIPTION

On aspect of the present disclosure is a first process for preparing a bio-based carbamate functionalized insecticide that includes one or more of performing a reductive amination reaction on an aldehyde to produce an amine product; and subjecting the amine product to a carbonylation reaction to produce the bio-based carbamate insecticide.

As used herein, the term “bio-based carbamate” refers to any carbamate functionalized active ingredient that is derived from renewable resources (e.g., cellulosic biomass containing lignosulfonates, kraft and/or alkali lignin). For example, a bio-based carbamate can be a carbamate synthesized from an aldehyde purified from the depolymerization of lignosulfonates. Carbamates derived from fossil fuel sources or fossil fuel derived chemicals (e.g., petroleum, crude oil, naphthalene, benzo hydrofurans, and some phenols) are not bio-based carbamates.

In exemplary embodiments, the bio-based carbamate functionalized insecticide is a compound of Formula (I):

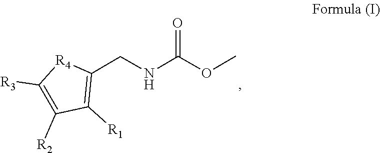

wherein:
R₁ is H,
R₂ is H,
R₃ is H, —CH₃, —CH₂Cl, CH₂Br, CH₂I, —CH₂OH, —COOH, or —CH₂OR₅, wherein R₅ is a C₁-C₆ alkyl chain, and
R₄ is O, S or NH.

In exemplary embodiments, the bio-based carbamate functionalized insecticide is a compound of Formula (I):

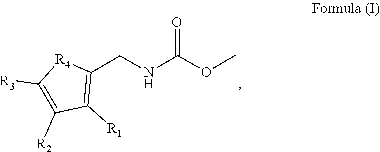

wherein:
R₁ is H,
R₂ is H,
R₃ is H, —CH₃, —CH₂Cl, —CH₂Br, —CH₂I or —CH₂OH, and
R₄ is O, S or NH.

In exemplary embodiments, the bio-based carbamate functionalized insecticide is a compound of Formula (I):

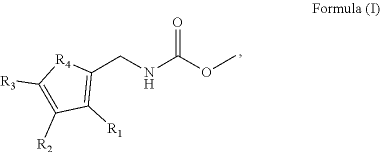

wherein:
R₁ is H,
R₂ is H,
R₃ is —CH₂OCH₃ or —COOH, and
R₄ is O, S or NH.

In exemplary embodiments, the bio-based carbamate functionalized insecticide is a compound of Formula (II):

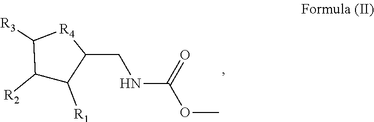

wherein:
R₁ is H,
R₂ is H,
R₃ is H, Cl, —CH₃, —CH₂OH, —CH₂Cl, —CH₂Br, —CH₂I, —COOH or —CH₂OR₅, wherein
R₅ is a C₁-C₆ alkyl chain, and
R₄ is O, S or NH.

In exemplary embodiments, the bio-based carbamate functionalized insecticide is a compound of Formula (II):

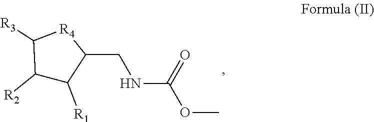

wherein:
R₁ is H,
R₂ is H,
R₃ is H, —CH₃, —CH₂Cl, —CH₂Br, —CH₂I or —CH₂OH, and
R₄ is O, S or NH.

In exemplary embodiments, the bio-based carbamate functionalized insecticide is a compound of Formula (II):

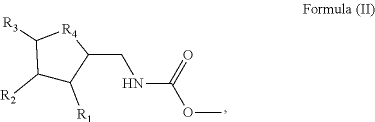

wherein:
R₁ is H,
R₂ is H,
R₃ is —CH₂OCH₃ or —COOH, and
R₄ is O, S or NH.

In exemplary embodiments, the bio-based carbamate functionalized insecticide is a compound of Formula (III)

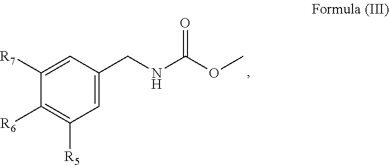

wherein:
R₅ is —OCH₃, —OH or H,
R₆ is —OCH₃, —OH or H, and
R₇ is —OCH₃, OH or H.

In exemplary embodiments, the bio-based carbamate functionalized insecticide is a compound with a structure of:

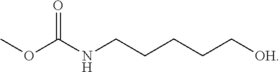

In exemplary embodiments, the aldehyde used in the reductive amination reaction is:

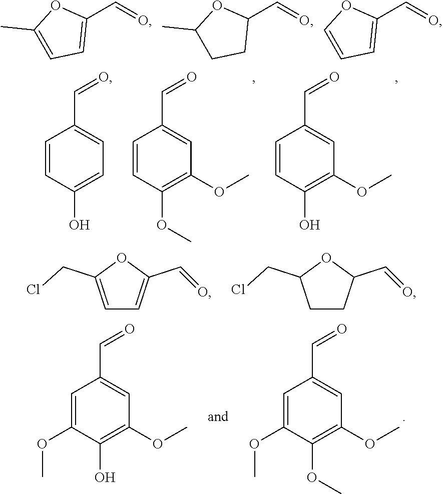

In exemplary embodiments, the aldehyde used in the reductive amination reaction is:

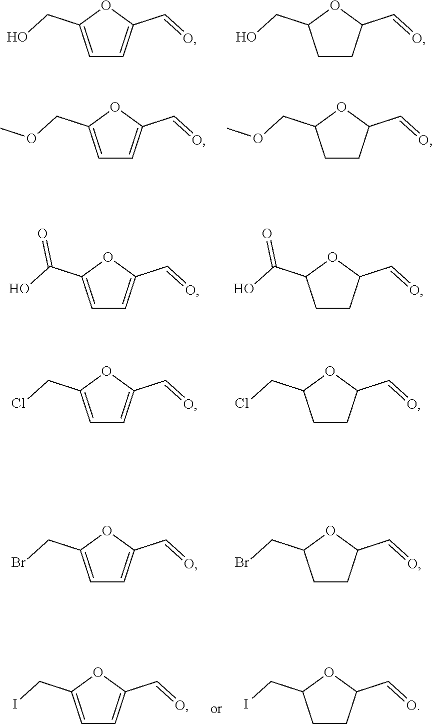

In exemplary embodiments, the amine product produced from the reductive amination reaction of the second process is:

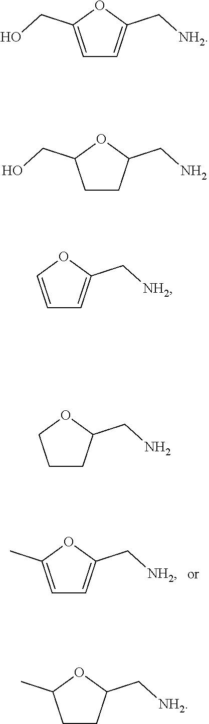

In exemplary embodiments, the performing of the reductive amination reaction in the first process includes reacting the aldehyde with ammonia under a H₂ pressure ranging from about 10 bar to about 100 bar, about 20 bar to about 80 bar, about 30 bar to about 70 bar, about 30 bar to about 60 bar, about 20 bar to about 70 bar, about 20 bar to about 50 bar or any pressure or pressure range falling within about 10 bar to about 100 bar. Those of ordinary skill will appreciate that the H₂ pressure can vary based on the equipment used to perform the reductive amination reaction and the scale of the reductive amination reaction. Accordingly, those of ordinary skill would be readily capable of determining the appropriate H₂ pressures needed to achieve production of the amine product resulting from the reductive amination reaction.

As used herein, the term “about” refers to a value that is ±5% of the stated value. In addition, it is understood that reference to a range of a first value to a second value includes the range of the stated values, e.g., a range of about 1 to about 5 also includes the more precise range of 1 to 5. It is also understood that the ranges disclosed herein include any selected subrange within the stated range, e.g., a subrange of about 50 to about 60 is contemplated in a disclosed range of about 1 to about 100.

In exemplary embodiments, the reductive amination reaction in the first process occurs for about 20 minutes to about 300 minutes, about 30 minutes to about 240 minutes, about 30 minutes to about 120 minutes, about 30 minutes to about 60 minutes or any time range falling with about 20 minutes to about 300 minutes.

In exemplary embodiments, the reductive amination reaction in the first process occurs for about 12 hours to about 24 hours, for about 12 hours to about 18 hours or for any amount of time falling within the range of about 12 hours to about 24 hours.

In exemplary embodiments, the reductive amination reaction in the first process is performed at a temperature ranging from about 70° C. to about 150° C., from about 80° C. to about 120° C., from about 80° C. to about 100° C. or any temperature range falling within the range of about 70° C. to about 150° C.

In exemplary embodiments, the reductive amination reaction in the first process is performed in a presence of a catalyst. The catalyst can be selected from, but is not limited to, Rh/Al₂O₃, Pd/C, Ru/C, Ru/ZrO₂, Raney Nickel, Rh/C or Pt/C.

In exemplary embodiments, the subjecting of the amine product to the carbonylation reaction in the first process includes reacting the amine product in a presence of a metal triflate catalyst. The metal triflate catalyst can be selected from, but is not limited to, ytterbium triflate, lanthanum triflate, scandium triflate or zinc triflate. An advantage of using metal triflate catalysts in the carbonylation reaction is that they can coordinate with carbonyl groups via their Lewis acidity, thereby promoting nucleophilic attack at the carbonyl carbon by the amine product produced from the reductive amination reaction. Accordingly, any metal catalyst that can coordinate with carbonyl groups to improve their electrophilicity can be used in the carbonylation reaction. Those of ordinary skill would be readily capable of determining the appropriate metal catalysts to use in the carbonylation reaction for improving the electrophilicity of the carbonyl carbon.

In exemplary embodiments, the carbonylation reaction in the first process is performed in the presence of dimethyl carbonate. Dimethyl carbonate can be present in a concentration ranging from about 20 mmol to about 100 mmol, about 20 mmol to about 60 mmol, about 60 mmol to about 100 mmol or any concentration falling within the range of about 20 mmol to 100 mmol. In exemplary embodiments, dimethyl carbonate is used as the solvent in the carbonylation reaction.

In exemplary embodiments, the carbonylation reaction in the first process is performed in the presence of methyl chloroformate. Methyl chloroformate can be present in a concentration ranging from about 20 mmol to about 100 mmol, about 20 mmol to about 60 mmol, about 60 mmol to about 100 mmol or any concentration falling within the range of about 20 mmol to 100 mmol. In exemplary embodiments, methyl chloroformate is used as the solvent in the carbonylation reaction.

In exemplary embodiments, the carbonylation reaction in the first process occurs for about 4 hours to about 24 hours, for about 8 hours to about 24 hours, for about 8 hours to about 16 hours or for any amount of time falling within the range of about 4 hours to about 24 hours.

In exemplary embodiments, the carbonylation reaction in the first process includes reacting the amine product with a catalyst (e.g., a metal triflate catalyst) in an amine product:catalyst ratio of about 16:1, about 15:1, about 14:1, about 13:1, about 12:1, about 11:1, or about 10:1.

In exemplary embodiments, the carbonylation reaction in the first process includes reacting the amine product with dimethyl carbonate or methyl chloroformate in a dimethyl carbonate or methyl chloroformate:amine product ratio of about 5:1, about 6:1, about 7:1, about 8:1, about 9:1, about 10:1, about 11:1, about 12:1, about 13:1, about 14:1, about 15:1, or about 20:1.

In exemplary embodiments, the carbonylation reaction in the first process includes reacting the amine product with dimethyl carbonate (DMC) or methyl chloroformate (MC) and a catalyst in a DMC or MC:amine:catalyst ratio of 10:1:0.1, 9:1:0.1, 8:1:0.1, 7:1:0.1, 6:1:0.1, 5:1:0.1, 10:1:0.5, 9:1:0.5, 8:1:0.5, 7:1:0.5, 6:1:0.5, 5:1:0.5 or 5-10:1:0.1-0.5.

In exemplary embodiments, the reductive amination reaction in the first process produces the amine product in a yield ranging from about 85% to about 100%, about 90% to about 99.9%, about 95% to about 99.9% or about 98% to about 99.9%.

In exemplary embodiments, the carbonylation reaction in the first process produces the bio-based carbamate functionalized insecticide in a yield ranging from 60% to about 95%, about 70% to about 95%, about 80% to about 95%, about 85% to about 95%, about 90% to about 95%. In exemplary embodiments, the carbonylation reaction in the first process produces the bio-based carbamate functionalized insecticide in a yield greater than 95%.

In exemplary embodiments, the first process includes isolating the aldehyde from a renewable resource containing lignosulfonates, hemicellulose, kraft and/or alkali lignin. Processes for isolating aldehydes and other compounds from renewable resources containing lignosulfonates, kraft and/or alkali lignin can be found in D. Bourbiaux et al. (D. Bourbiaux, J. Pu, F. Rataboul, L. Djakovitch, C. Geantet, D. Laurenti, Catal. Today 2021, 373, 24-37), Fache et al. (M. Fache, B. Boutevin, S. Caillol, ACS Sustain. Chem. Eng. 2015, 4, 35-36), Pacek et al. (A. W. Pacek, P. Ding, M. Garrett, G. Sheldrake, A. W. Nienow, Ind. Eng. Chem. Res. 2013, 52, 8361-8372) and Wang et al. (Y. Wang, S. Sun, F. Li, X. Cao, R. Sun, Ind. Crops Prod. 2018, 116, 116-121). In exemplary embodiments, isolating of the aldehyde from the renewable resource includes one or more of depolymerizing lignosulfonates to produce a reaction mixture containing the aldehyde and precipitating the aldehyde out of the reaction mixture.

In exemplary embodiments, the renewable resource is a cellulosic biomass. As used herein, the term “cellulosic biomass” refers to virtually any plant-derived organic matter (woody or non-woody) available for energy on a sustainable basis, such as, but not limited to plant biomass. Plant biomass can include, but is not limited to, agricultural crop wastes and residues such as corn stover, aquatic plants (e.g., algae), wheat straw, rice straw, sugar cane bagasse, woody energy crops, wood wastes, residues from trees such as fruit trees and fruit-bearing trees (e.g., apple trees and orange trees), softwood forest thinnings, barky wastes, sawdust, paper and pulp industry waste streams, wood fiber, grass crops, prairie cord grass, switchgrass, miscanthus, big bluestem, little bluestem, side oats grama, yard waste (e.g., grass clippings, leaves, tree clippings, brush, etc.) and vegetable processing waste.

Another aspect of the present disclosure is a second process for preparing a bio-based carbamate functionalized insecticide that includes at least one or more of performing a reductive amination reaction on an aldehyde to produce an amine product; subjecting the amine product to an aminolysis reaction to produce the bio-based carbamate functionalized insecticide, optionally performing a ring hydrogenation reaction on the produced carbamate functionalized insecticide, optionally performing an etherification reaction on the produced carbamate functionalized insecticide to produce an ether-containing bio-based carbamate functionalized insecticide, and/or optionally performing a ring hydrogenation on the ether-containing bio-based carbamate functionalized insecticide.

In exemplary embodiments, the aldehyde of the second process is an aldehyde of the first process. In exemplary embodiments, the aldehyde of the second process is:

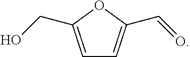

In exemplary embodiments, the amine product produced from the reductive amination reaction of the second process is an amine product produced from the reductive amination reaction of the first process.

In exemplary embodiments, the amine product produced from the reductive amination reaction of the second process is:

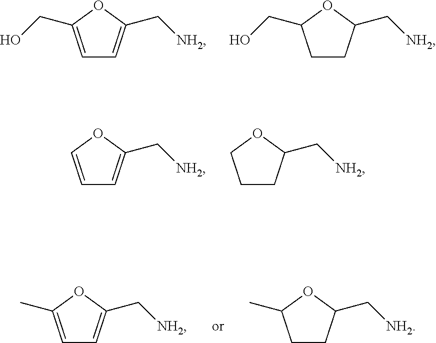

In exemplary embodiments, the performing of the reductive amination reaction in the second process includes reacting the aldehyde with ammonia under a H₂ pressure ranging from about 10 bar to about 100 bar, about 20 bar to about 80 bar, about 30 bar to about 70 bar, about 30 bar to about 60 bar, about 20 bar to about 70 bar, about 20 bar to about 50 bar or any pressure or pressure range falling within about 10 bar to about 100 bar. Those of ordinary skill will appreciate that the H₂ pressure can vary based on the equipment used to perform the reductive amination reaction and the scale of the reductive amination reaction. Accordingly, those of ordinary skill would be readily capable of determining the appropriate H₂ pressures needed to achieve production of the amine product resulting from the reductive amination reaction.

In exemplary embodiments, the reductive amination reaction in the second process occurs for about 20 minutes to about 300 minutes, about 30 minutes to about 240 minutes, about 30 minutes to about 120 minutes, about 30 minutes to about 60 minutes or any time range falling with about 20 minutes to about 300 minutes.

In exemplary embodiments, the reductive amination reaction in the second process occurs for about 12 hours to about 24 hours, for about 12 hours to about 18 hours or for any amount of time falling within the range of about 12 hours to about 24 hours.

In exemplary embodiments, the reductive amination reaction in the second process is performed at a temperature ranging from about 70° C. to about 150° C., from about 80° C. to about 120° C., from about 80° C. to about 100° C. or any temperature range falling within the range of about 70° C. to about 150° C.

In exemplary embodiments, the reductive amination reaction in the second process is performed in a presence of a catalyst. The catalyst can be selected from, but is not limited to, Rh/Al₂O₃, Pd/C, Ru/C, Ru/ZrO₂, Raney Nickel, Rh/C or Pt/C.

In exemplary embodiments, the reductive amination reaction in the second process produces the amine product in a yield ranging from about 85% to about 100%, about 90% to about 99.9%, about 95% to about 99.9% or about 98% to about 99.9%.

In exemplary embodiments, the aminolysis reaction of the second process is conducted in the presence of a base. The base can be selected from one that is a strong proton acceptor (high basicity) but a poor electron donor to electrophiles. Examples of possible bases that can be present during the aminolysis reaction include, but are not limited to, triethylamine (Et₃N), inorganic bases (such as sodium hydroxide (NaOH), potassium carbonate (K₂CO3), sodium carbonate (Na₂CO₃)), pyridine, DMAO (4-methylaminopyridine), imidazole, morpholine, lithium diisopropylamide (LDA), N,N-diisopropylethylamine (DIPEA), potassium tert-butoxide, 1,8-diazabicycloundec-7-ene (DBU) and 1,5-diazabicyclo(4.3.0)non-5-ene (DBN). The molar ratio of the non-nucleophilic base to the amine product during the aminolysis reaction can range from about 2.5:1 to about 1:1.

In exemplary embodiments, the aminolysis reaction of the second process includes mixing the amine product with a solvent to produce a reaction mixture and cooling the reaction mixture to a temperature of about 15° C. or lower, about 10° C. or lower, about 5° C. or lower, or about 0° C. or lower. The solvent can be any solvent capable of solubilizing the amine product. Examples of possible solvents include, but are not limited to, dichloromethane, ethyl acetate, tetrahydrofuran and toluene.

In exemplary embodiments, the aminolysis reaction of the second process includes reacting the amine product with an aminolysis reagent capable of converting the amine function group on the amine product to a carbamate functional group. The aminolysis reagent can be selected from, but is not limited to, acid chlorides (e.g., methyl chloroformate), dialkyl carbonates (e.g., dimethyl carbonate), alkyl isocyanates, esters or any reagent known in the art to be capable of converting an amine functional group to a carbamate functional group. Those of ordinary skill in the art, with the benefit of the present disclosure in combination with their general understanding of the art, are readily capable of determining what other reagents could be used as the aminolysis reagent when considering the structure of the amine product and the conditions of the aminolysis reaction. The molar ratio of the aminolysis reagent to the amine product during the aminolysis reaction can range from about 1.5:1 to about 1:1.

In exemplary embodiments, the aminolysis reaction of the second process occurs for about 10 minutes to about 300 minutes, for about 20 minutes to about 300 minutes, about 30 minutes to about 240 minutes, about 30 minutes to about 120 minutes, about 30 minutes to about 60 minutes or any time range falling with about 20 minutes to about 300 minutes.

In exemplary embodiments, the bio-based carbamate functionalized insecticide produced from the aminolysis reaction of the second process is selected from:

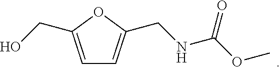

In exemplary embodiments, the bio-based carbamate functionalized insecticide produced from the aminolysis reaction of the second process is a compound of Formula (I):

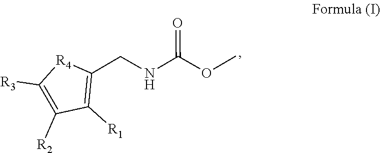

wherein:
R₁ is H,
R₂ is H,
R₃ is H, —CH₃, —CH₂Cl, —CH₂Br, —CH₂OH, —COOH or —CH₂OR₅, wherein R₅ is a C₁-C₆ alkyl chain, and
R₄ is O, S or NH.

In exemplary embodiments, the second process includes performing a ring hydrogenation reaction of the bio-based carbamate functionalized insecticide produced from the aminolysis reaction to produce a bio-based carbamate functionalized insecticide having the following structure:

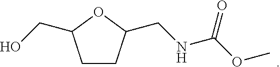

In exemplary embodiments, the second process includes performing a ring hydrogenation reaction of the bio-based carbamate functionalized insecticide produced from the aminolysis reaction to produce a bio-based carbamate functionalized insecticide having the following structure of Formula (II):

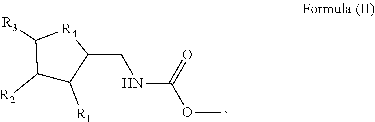

wherein:
R₁ is H,
R₂ is H,
R₃ is H, —CH₃, —CH₂Cl, —CH₂Br, —CH₂I, —CH₂OH, —COOH or —CH₂OR₅, wherein R₅ is a C₁-C₆ alkyl chain, and
R₄ is O, S or NH.

In exemplary embodiments, the second process includes performing an etherification reaction on the bio-based carbamate functionalized insecticide produced from the aminolysis reaction to produce an ether-containing bio-based carbamate functionalized insecticide.

In exemplary embodiments, the etherification reaction includes reacting the bio-based carbamate functionalized insecticide produced from the aminolysis reaction with an etherification reagent capable of converting an alcohol group to an ether group to produce the ether-containing bio-based carbamate functionalized insecticide. The etherification reagent can be selected from, but is not limited to, alkyl halides (e.g., methyl iodide), alkoxides (e.g., sodium alkoxide) or any other reagent known in the art to be capable of converting an alcohol functional group to an ether functional group. Those of ordinary skill in the art, with the benefit of the present disclosure in combination with their general understanding of the art, are readily capable of determining what other reagents could be used as the etherification reagent when considering the structure of the bio-based carbamate functionalized insecticide produced from the aminolysis reaction and the conditions of the etherification reaction. The molar ratio of the etherification reagent to the bio-based carbamate functionalized insecticide produced from the aminolysis reaction during the etherification reaction can range from about 1.5:1 to about 1:1.

In exemplary embodiments, the etherification reaction is conducted in the presence of a base. Possible examples of bases that can be included in the etherification reaction include, but are not limited to, those that are strong proton acceptors (high basicity) but poor electron donors to electrophiles, such as lithium diisopropylamide (LDA), N,N-diisopropylethylamine (DIPEA), and potassium tert-butoxide, those that contain electronic structures capable of delocalizing a negative charge, such as 1,8-diazabicycloundec-7-ene (DBU) and 1,5-diazabicyclo(4.3.0)non-5-ene (DBN), and strong bases such as potassium hydroxide and sodium alkoxides, such as NaOEt and NaOMe.

In exemplary embodiments, the etherification reaction of the second process includes mixing bio-based carbamate functionalized insecticide produced from the aminolysis reaction with a solvent to produce a reaction mixture. The solvent can be any solvent capable of solubilizing the bio-based carbamate functionalized insecticide produced from the aminolysis reaction. Examples of possible solvents include, but are not limited to, dichloromethane, ethyl acetate, tetrahydrofuran, toluene, alcohols (e.g., methanol and ethanol), dimethyl Sulfoxide (DMSO), and N, N-Dimethylformamide (DMF).

In exemplary embodiments, the ether-containing bio-based carbamate functionalized insecticide has the following structure:

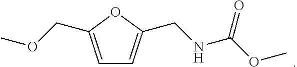

In exemplary embodiments, the ether-containing bio-based carbamate functionalized insecticide has a structure according to Formula (IV):

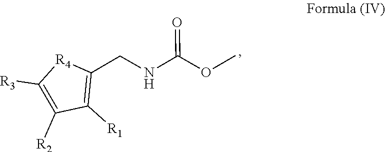

wherein:
R₁ is H,
R₂ is H,
R₃ is —CH₂OR₅, wherein R₅ is a C₁-C₆ alkyl chain, and
R₄ is O, S or NH.

In exemplary embodiments, the second process includes performing a ring hydrogenation on the ether-containing bio-based carbamate functionalized insecticide to create a bio-based carbamate functionalized insecticide having the following structure:

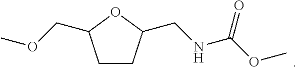

In exemplary embodiments, the second process includes performing a ring hydrogenation on the ether-containing bio-based carbamate functionalized insecticide to create a bio-based carbamate functionalized insecticide having the structure of Formula (V):

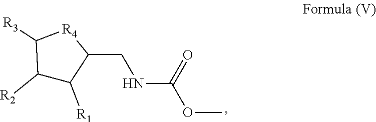

wherein:
R₁ is H,
R₂ is H,
R₃ is —CH₂OR₅, wherein R₅ is a C₁-C₆ alkyl chain, and
R₄ is O, S or NH.

In exemplary embodiments, the ring hydrogenation reactions of the second process includes subjecting the ether-containing bio-based carbamate functionalized insecticide and/or the bio-based carbamate functionalized insecticide produced from the aminolysis reaction to a hydrogenation catalyst and a hydrogen source. The hydrogenation catalyst can be selected from, but is not limited to, Pd/C catalysts, Pt/C catalysts, Ru/C catalysts, Raney Nickel, Platinum oxide or any other catalyst known in the art to be capable of reducing double bonds. Those of ordinary skill in the art, with the benefit of the present disclosure in combination with their general understanding of the art, are readily capable of determining what other catalysts can be used when considering the structure of the bio-based carbamate functionalized insecticides. The hydrogen source can be selected from, but is not limited to, alcohols (e.g., isopropanol), molecular H₂ gas, formic acid and formates, or any other hydrogen sources known in the art to be capable of donating hydrogen for the reduction of double bonds. Those of ordinary skill in the art, with the benefit of the present disclosure in combination with their general understanding of the art, are readily capable of determining what other hydrogen sources can be used when considering the structure of the bio-based carbamate functionalized insecticides.

Another aspect of the present disclosure is a third process for preparing a bio-based carbamate functionalized insecticide that includes at least one or more of performing an azidation reaction on a methyl-ester containing an alkyl halogen group to produce an azide intermediate, subjecting the azide intermediate to an azide reduction reaction to produce an amine intermediate, performing an aminolysis reaction on the amine intermediate to produce a methyl ester carbamate intermediate, subjecting the methyl ester carbamate intermediate to a hydrolysis reaction to produce the bio-based carbamate functionalized insecticide, and/or optionally performing a ring hydrogenation on the bio-based carbamate functionalized insecticide.

In exemplary embodiments, the methyl-ester containing the alkyl halogen group has a structure selected from:

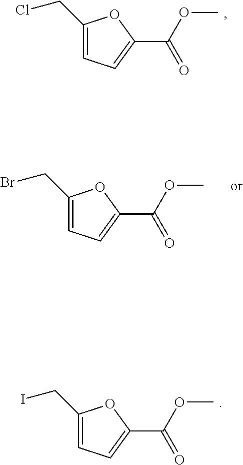

In exemplary embodiments of the third process, the azidation reaction is performed on either an ethyl ester containing an alkyl halogen group, a propyl ester containing an alkyl halogen group, an isopropyl ester containing an alkyl halogen group or a tert-butyl ester containing an alkyl halogen group instead of the methyl-ester containing an alkyl halogen group.

In exemplary embodiments of the third process, the azidation reaction is performed on an ester having a structure of Formula (VI):

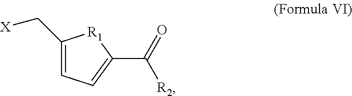

wherein:
R₁ is O, S or NH,
R₂ is OR₃, wherein R₃ is a C₁-C₆ alkyl chain, and
X is a halogen.

In exemplary embodiments, the azidation reaction of the third process includes reacting the methyl-ester containing the alkyl halogen group with an azidation agent capable of converting a halogen functional group into an azide (N₃) functional group. The azidation agent can be selected from, but is not limited to, sodium azide, hydrazoic acid, azidotrimethylsilane, iodine azide, imidazole-1-sulfonyl azide hydrochloride or any other azidation agent known in the art to be capable of converting a halogen functional group into an azide functional group. Those of ordinary skill in the art, with the benefit of the present disclosure in combination with their general understanding of the art, are readily capable of determining what other reagents could be used as the azidation agent when considering the structure of the methyl-ester containing the alkyl halogen group and the conditions of the azidation reaction. The molar ratio of the azidation agent to the methyl-ester containing the alkyl halogen group during the azidation reaction can range from about 2.0:1 to about 1:1

In exemplary embodiments, the azidation reaction of the third process is conducted in a solvent mixture containing one or more of the following solvents: methanol, water, DMF, DMSO, acetonitrile, tetrahydrofuran, other alcohols (e.g., ethanol or propanol), dioxane or any other solvent known in the art to be used for azidation reactions.

In exemplary embodiments, the azidation reaction includes creating a reaction mixture containing the azidation agent, the methyl-ester containing the alkyl halogen group, and the solvent mixture; and mixing these components together at a temperature ranging from about 50° C. to about 150° C., from about 60° C. to about 120° C., from about 65° C. to about 100° C. or any temperature range falling within the range of about 50° C. to about 150° C. In exemplary embodiments, the mixing occurs for about 30 minutes to about 24 hours, about 1 hour to about 12 hours, about 2 hours to about 4 hours, or about any amount of time falling within the range of about 30 minutes to about 24 hours.

In exemplary embodiments, the azide intermediate has a structure selected from:

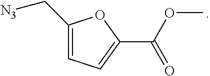

In exemplary embodiments, the azide intermediate has a structure of Formula (VII):

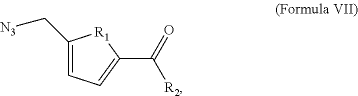

wherein:
R₁ is O, S or NH, and
R₂ is OR₃, wherein R₃ is a C₁-C₆ alkyl chain.

In exemplary embodiments, the azide reduction reaction of the third process includes mixing the azide intermediate with an acid and an reduction catalyst in a solvent at a temperature ranging from 20° C. to about 60° C., about 25° C. to about 40° C. or any temperature falling within the range of 20° C. to about 60° C. for about 2 hours to about 24 hours, 4 hours to 16 hours, 6 hours to 8 hours or any timepoint falling within the range of about 2 hours to about 24 hours to produce the amine intermediate. The solvent can be selected from, but is not limited to, alcohols (e.g., methanol, ethanol), water, ethyl acetate, toluene, chloroform, and tetrahydrofuran. The acid can be selected from, but is not limited to, hydrochloric acid, sulfuric acid, or other organic or inorganic acids. The reduction catalyst can be selected from, but is not limited to, Pd/C, Raney Nickel, Platinum, copper catalysts (e.g., those used in azide-alkyne click reactions), iron catalysts (e.g., Fe(OAc)₂ and Fe(OTf)₂), or any other catalytic materials that are known in the art to be used for azide reduction reactions. In exemplary embodiments, the mixing of the azide intermediate with the acid and the reduction catalyst occurs in the presence of H₂ gas.

In exemplary embodiments, the amine intermediate has a structure selected from:

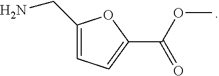

In exemplary embodiments, the amine intermediate has a structure of Formula (VIII):

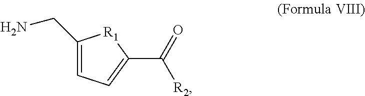

wherein:
R₁ is O, S or NH, and

R₂ is OR₃, wherein R₃ is a C₁-C₆ alkyl chain.

In exemplary embodiments, the aminolysis reaction of the third process is conducted in the presence of a base. The base can be selected from one that is a strong proton acceptor (high basicity) but a poor electron donor to electrophiles. Examples of possible bases that can be present during the aminolysis reaction include, but are not limited to, triethylamine (Et₃N), inorganic bases (such as sodium hydroxide (NaOH), potassium carbonate (K₂CO3), sodium carbonate (Na₂CO₃)), pyridine, DMAO (4-methylaminopyridine), imidazole, morpholine, lithium diisopropylamide (LDA), N,N-diisopropylethylamine (DIPEA), potassium tert-butoxide, 1,8-diazabicycloundec-7-ene (DBU) and 1,5-diazabicyclo(4.3.0)non-5-ene (DBN). The molar ratio of the non-nucleophilic base to the amine intermediate during the aminolysis reaction can range from about 2.5:1 to about 1:1.

In exemplary embodiments, the aminolysis reaction of the third process includes mixing the amine intermediate with a solvent to produce a reaction mixture and cooling the reaction mixture to a temperature of about 15° C. or lower, about 10° C. or lower, about 5° C. or lower, or about 0° C. or lower. The solvent can be any solvent capable of solubilizing the amine intermediate. Examples of possible solvents include, but are not limited to, dichloromethane, ethyl acetate, tetrahydrofuran and toluene.

In exemplary embodiments, the aminolysis reaction of the third process includes reacting the amine intermediate with an aminolysis reagent capable of converting the amine functional group on the amine intermediate to a carbamate functional group. The aminolysis reagent can be selected from, but is not limited to, alkyl chloroformates (e.g., methyl chloroformate), dialkyl carbonates (e.g., dimethyl carbonate), alkyl isocyanates, alkyl esters or any reagent known in the art to be capable of converting an amine functional group to a carbamate functional group. Those of ordinary skill in the art, with the benefit of the present disclosure in combination with their general understanding of the art, are readily capable of determining what other reagents could be used as the aminolysis reagent when considering the structure of the amine product and the conditions of the aminolysis reaction. The molar ratio of the aminolysis reagent to the amine intermediate during the aminolysis reaction can range from about 2.5:1 to about 1:1.

In exemplary embodiments, the aminolysis reaction of the third process occurs for about 10 minutes to about 300 minutes, for about 20 minutes to about 300 minutes, about 30 minutes to about 240 minutes, about 30 minutes to about 120 minutes, about 30 minutes to about 60 minutes or any time range falling with about 20 minutes to about 300 minutes.

In exemplary embodiments, the methyl ester carbamate intermediate has a structure selected from:

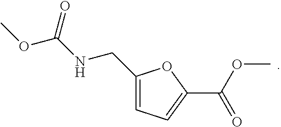

In exemplary embodiments, the aminolysis reaction of the third process creates an ester carbamate intermediate having a structure of Formula IX:

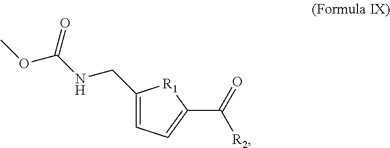

wherein:
R₁ is O, S or NH, and
R₂ is OR₃, wherein R₃ is a C₁-C₆ alkyl chain.

In exemplary embodiments, the hydrolysis reaction of the third process includes mixing the methyl ester carbamate intermediate with a nucleophilic base in a solvent system. The nucleophilic base can be selected from lithium hydroxide, sodium hydroxide, potassium hydroxide or any other base that is capable of producing a nucleophilic hydroxide ion in the solvent system. The solvent system can include one or more of the following solvents: tetrahydrofuran, methanol, ethanol, propanol, butanol and water.

In exemplary embodiments, the hydrolysis reaction of the third process includes mixing the ester carbamate intermediate having a structure of Formula IX with a nucleophilic base in the solvent system.

In exemplary embodiments, the bio-based carbamate functionalized insecticide produced from the hydrolysis reaction has a structure selected from:

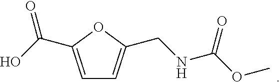

In exemplary embodiments, the bio-based carbamate functionalized insecticide produced from the hydrolysis reaction has a structure of Formula X:

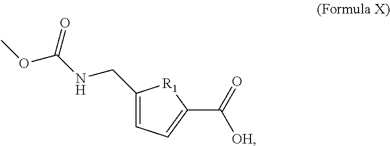

wherein:
R₁ is O, S or NH.

In exemplary embodiments, the bio-based carbamate functionalized insecticide produced from the hydrolysis reaction of the third process is subjected to a ring hydrogenation. In exemplary embodiments, the ring hydrogenation reaction of the third process includes subjecting the bio-based carbamate functionalized insecticide produced from the hydrolysis reaction to a hydrogenation catalyst and a hydrogen source. The hydrogenation catalyst can be selected from, but is not limited to, Pd/C catalysts, Pt/C catalysts, Raney Nickel, platinum oxide or any other catalyst known in the art to be capable of reducing double bonds. Those of ordinary skill in the art, with the benefit of the present disclosure in combination with their general understanding of the art, are readily capable of determining what other catalysts can be used when considering the structure of the bio-based carbamate functionalized insecticide produced from the hydrolysis reaction. The hydrogen source can be selected from, but is not limited to, alcohols (e.g., isopropanol), molecular H₂ gas, formic acid and formates, or any other hydrogen sources known in the art to be capable of donating hydrogen for the reduction of double bonds. Those of ordinary skill in the art, with the benefit of the present disclosure in combination with their general understanding of the art, are readily capable of determining what other hydrogen sources can be used when considering the structure of the bio-based carbamate functionalized insecticide produced from the hydrolysis reaction.

In exemplary embodiments, the bio-based carbamate functionalized insecticide produced from the ring hydrogenation reaction of the third process has a structure of Formula XI:

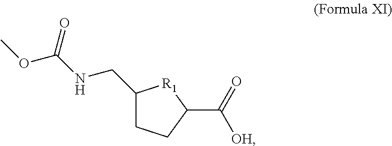

wherein:
R₁ is O, S or NH.

Another aspect of the present disclosure is an insecticidal composition including at least one bio-based carbamate functionalized insecticide produced from any one of the processes disclosed herein.

The insecticidal composition can be used to repel one or more target pest such as, but not limited to, animals belonging to the animal order of Acari, Anoplura, Araneae, Blattodea, Coleoptera, Collembola, Diptera, Grylloptera, Heteroptera, Homoptera, Hymenoptera, Isopoda, Isoptera, Lepidoptera, Mantodea, Mallophaga, Neuroptera, Odonata, Orthoptera, Psocoptera, Siphonaptera, Symphyla, Thysanura, and/or Thysanoptera.

In exemplary embodiments, the composition has an LC₅₀ against adult Alphitobius diaperinus of at least 6,660,622 ng/cm³.

In exemplary embodiments, the insecticidal composition comprises one or more additives selected from, but not limited to, botanical essential oils or concentrates (e.g., camphor oil, Litsea cubeba oil, spearmind oil, citronella oil, geranial oil, cassia oil, star anise oil, cedar wood oil, peppermint oil, wintergreen oil and/or orris concrete), surfactants (e.g., ethanolamine dodecyl sulfate (K12 EA), sodium dodecyl sulfate (AS), potassium dodecyl phosphate (PK), linear sodium alkyl benzene sulfonate (LAS), sodium oleoyl amino fatty acid, sodium N-oleoyl-N-methyl taurinate, sodium salt α-sulfo fatty acid methyl ester (MES), sodium stearate, sodium oleate, ammonium oleate, glycerol monooleate, potassium oleate, potassium stearate, zinc stearate, magnesium stearate, polyoxyethylene fatty alcohol sodium sulfate (AES), ethoxylated alkyl ester sulfo succinate, alkylbenzene sulfonic acid, sodium alkyl sulfonate, a-alkene-sulfonate (AOS), secondary alkane sulfonate (SAS), sodium dialkyl ester sulfonsuccinate, N-acyl glutamate (AGA), triethanolamine polyoxyethylene fatty alcohol sulfate (TA-40), sulfonated caster oil, Span-20, 40, 60, 65, 80, Tween-20, 40, 60, 65, 80, secondary alcohol polyoxyethylene ether (JFC), fatty alcohol polyoxyethylene (3) ether (AE03), fatty alcohol polyethoxylate (7) ether (AE07), fatty alcohol polyoxyethylene (9) ether (AE09), fatty alcohol polyethoxylate(IO) ether (AEO10), fatty alcohol Polyoxyethylene (15) ether (AE015), fatty acid alkanol amide, fatty acid polyoxyethylene (IO) ester, glyceryl oleate, glycerol trioleate, glyceryl stearate, alkylphenol polyoxyethylene (IO) ether, coco fatty diethanol amide, coco fatty monoethanol amide, caster oil polyoxyethylene ether, ethoxylated metluyl glucoside sesquistearate (MSE), methyl glucoside sesquistearate, sucrose fatty acid ester, polyoxyethylene-polyoxypropylene glycols, polyoxyethylene alkylamide, dodecyl dimethyl betaine, alkyl dimethyl betaine, coco amino propyl betaine, carboxylate-type imidazoline ampholytic surfactant, carbomer, ethoxylated lanoilu, ethoxylated lanoilu alcohol, lanolin fatty acid, iso-propyl lanolate, liquid lanolin, and lecithin), synergists (e.g., alkyl glucoside, lactic acid, methyl silicone oil, isopropyl alcohol, ethanol, oleic acid, polyoxyethylene alkyl ether, alkyl sulfates, dialkyl succinate, alkyl amide taurine salt, fatty alcohol polyoxyethylene ether sulfonate, fatty alcohol ethoxylates, lactic acid ethyl ester, phenethyl propionate, polyglyceryl stearate, sodium polyacrylate, calcium lignin sulfonate, dialkyl succinate sulfonate, ethylene glycol monoether, amyl acetate, 2-butanone, n-butyl alcohol, dibutyl phthalate and n-butyl acetate), terpenes and/or terpenoids.

In exemplary embodiments, the composition contains one or more additives in a weight percentage, based on the total weight of the composition, of about 1 wt %, about 5 wt %, about 10 wt %, about 20 wt %, about 30 wt %, about 40 wt %, about 50 wt %, about 60 wt %, about 70 wt %, about 80 wt % or any weight percentage or weight percentage range falling within the range of 1 wt % to 80 wt %.

The insecticidal composition can be in the form of an emulsion, a powder, a water-based spray or an aerosol.

In exemplary embodiments, the composition contains the at least one bio-based carbamate functionalized insecticide in a weight percentage, based on the total weight of the composition, of about 1 wt %, about 5 wt %, about 10 wt %, about 20 wt %, about 30 wt %, about 40 wt %, about 50 wt %, about 60 wt %, about 70 wt %, about 80 wt % or any weight percentage or weight percentage range falling within the range of 1 wt % to 80 wt %.

### EXAMPLES

The present disclosure will be described in more detail with reference to the following Examples, which shows exemplary embodiments in accordance with the present disclosure. The present disclosure is not limited to these exemplary embodiments.

### Example 1: Bio-Based Insecticide Production from Biomass Derived Furfural and Vanillin

### I. Materials and Methods of Example 1

### Materials

Methylene chloride, furfural, vanillin, furfurylamine, anhydrous sodium sulfate, anhydrous dimethyl carbonate, liquid ammonia, deuterated solvents d₆-DMSO, and CDCl₃ were all used as received. Supported metal oxides and metal triflate catalysts were all purchased from Sigma Aldrich. Metal oxides were reduced at 300° C. for 3 hours at a ramp rate of 10° C./min under 100 cm³/min 10% hydrogen/helium flow. Vanillylamine was purchased from MedChem.

### Catalytic Reaction and Product Analysis

The reductive amination reactions were conducted in 50 mL Parr reactors. The reactants and the catalyst were placed in a glass liner with a magnetic stir bar. After loading the glass liner in the Parr reactor, the reactor was closed, purged three times with nitrogen, twice with hydrogen, and then pressurized to the desired hydrogen pressure. After a designated reaction time, the reactor was cooled in an ice bath, depressurized, and opened. Products were extracted using methylene chloride with a gravity separation funnel.

For the next carbonylation step, reactions were conducted in 10 mL glass vials heated in an aluminum heating block with controlled stirring. Scale-up reactions were conducted in 50 mL round bottom flasks heated in an oil bath and connected to a reflux condenser. Temperature and stirring rate were maintained at 80° C. and 800 rpm, respectively. After reacting for a set amount of time, the reaction vials were removed from the heating block and cooled to room temperature. The products and remaining reactants were identified and quantified via a gas chromatogram mass spectrometer (GC-MS) equipped with a DB-5 column and a gas chromatogram with a flame ionization detector (GC-FID) equipped with an HP-1 column. For quantification, the effective carbon number method (ECN) was used, with eicosane as the internal standard (J. T. Scanlon, D. E. Willis, J. Chromatogr. Sci., 1985, 23, 333-340). Calibration curves were developed whenever compounds were either commercially available, such as furfural, vanillin, furfurylamine, vanillylamine or were purified in large quantities, for example methyl (2-furylmethyl) carbamate (FC) and methyl (4-hydroxy-3-methoxybenzyl) carbamate (VC).

### Reductive Amination

Upon completion of the reductive amination reactions, the Rh/Al₂O₃ catalyst was recovered through vacuum filtration. The collected catalyst was washed multiple times with DI (de-ionized) water and re-used for subsequent reactions.

### Carbonylation

Upon completion of the carbonylation reaction, dimethyl carbonate was removed through rotary evaporation and the residue was dissolved in 20 mL of methylene chloride. The organic solution was subjected to three washes with 20 mL of DI water, the aqueous phases were collected, and the water was distilled off to recover the lanthanum triflate catalyst. The triflate was washed with methylene chloride to remove residual organic compounds and air-dried before re-use.

### Product Purification and Characterization

Upon completion of the carbonylation reaction, the organic solution was diluted with methylene chloride and subjected to multiple washes with 2% citric acid to purify the amine and extract any remaining triflate catalyst in the organic solution. The organic phase was then dried over anhydrous sodium sulfate, and the pure carbamate was obtained via rotary evaporation.

The carbamate products were characterized to determine purity and molecular weight. Liquid chromatography-mass spectrometry (LC-MS) and electron spray ionizations mass spectrometry (ESI-MS) data was obtained from the Mass Spectrometry Facility at the University of Delaware. All reported results were done in duplicates.

### Lethal Concentration Studies

The median lethal concentration (LC₅₀) of chemicals was measured in adult lesser mealworm beetles (Alphitobius diaperinus) through vial bioassays. Adult lesser mealworm beetles are a common pest worldwide and often used as a test species for commercial insecticides. The beetles were sourced from a UD Agricultural Entomology Lab colony collected from a Delaware broiler house and housed in a controlled growth chamber (kept at 25° C., ˜60% humidity, and in darkness). The colony was sustained on chicken layer pellets, supplemented with apple and carrot, and with Styrofoam blocks provided as pupation substrate. Preliminary screenings determined lethal concentration ranges for each chemical, achieved in glass vials by evaporating acetone-based insecticide dilutions. Five replicates per concentration and acetone-only controls were set up for each chemical. Mortality and morbidity assessments were conducted after 48-hour exposures. Statistical analysis involved probit regression, excluding vials displaying 0% or 100% mortality to avoid influences of false endpoints. Adjustments for heterogeneity factors were made based on chi-square goodness-of-fit test results.

### Computational Ecotoxicity Simulations

The Toxicity Estimation Software Tool (TEST), developed by EPA, was used to estimate the lethal dose for 50% death (LD₅₀) for mammals and lethal concentration (LC₅₀) for aquatic species. The species considered were a rat, fathead minnow (fish), and Daphnia magna (plankton). TEST is a peer-reviewed tool using QSAR (Quantitative Structure-Activity Relationship) to estimate chemical toxicity based on molecular descriptors, like molecular weight and octanol-water partition coefficient (log(K_{ow})). TEST has been trained on datasets from ECOTOX, which contain various aliphatic and aromatic carbamates, amines, and amides structurally similar to the carbamates analyzed in this Example. TEST predictions were supplemented by ECOSAR (J. T. R. Tracy Wright et al., “Operation manual for the ecolological Structure-Activity Relationship Model (ECOSAR) Class Program, 2022; and H. Sanderson, et al., Toxicol. Lett., 2003, 144, 383-395). ECOSAR estimates acute toxicity of aquatic species using the Meyer-Overton relationship, applied to chemicals within a structurally similar class. ECOSAR has been optimized for molecules with a log P less than 5 and molecular weight less than 1000 g/mol. Biotransformer was used to predict small molecule metabolism in human tissues, gut, and the environment.

### Hydrolysis Degradation Experiments

Hydrolysis was done by dissolving 100 mg of the carbamate compounds in methanol and adding the solution to an alkaline buffer. The organic phase of these solutions was extracted with ethyl acetate after 24 hours and analyzed using GC-MS.

### ASPEN Simulations

The capital and operating costs are estimated using the Aspen Process Economic Analyzer (APEA) V12.0 (“Aspen Plus V12,” DOI 10.1007/BF00447266, 2023). The economic evaluation was conducted based on the discounted cash flow analysis. The number of years for analysis was 20 and the payout time was 10. The active ingredient's minimum selling price (MSP) was determined such that the net present value (NPV) was zero at payout time. The cost of catalyst recovery was not considered.

### II. Results of Example 1 Experiments

### Reductive Amination of Vanillin

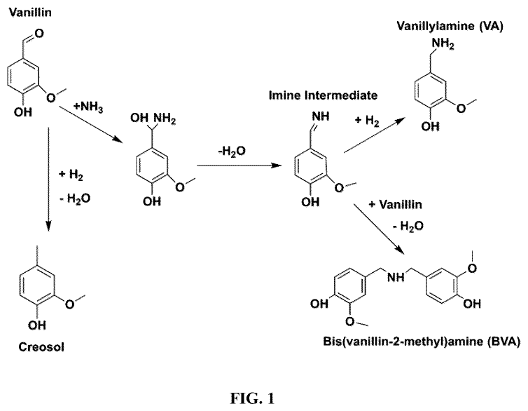

The selective reductive amination for vanillin was challenging due to its propensity to dimerize. FIG. 1 illustrates a reaction network for the reductive amination for vanillin. Creosol (a byproduct) results from the hydrodeoxygenation of vanillin. Reductive amination progressed through a Schiff base-type imine intermediate from the interaction of ammonia and the aldehyde group. The Schiff base-type imine intermediate then underwent a hydrogenation to vanillylamine or a secondary amine.

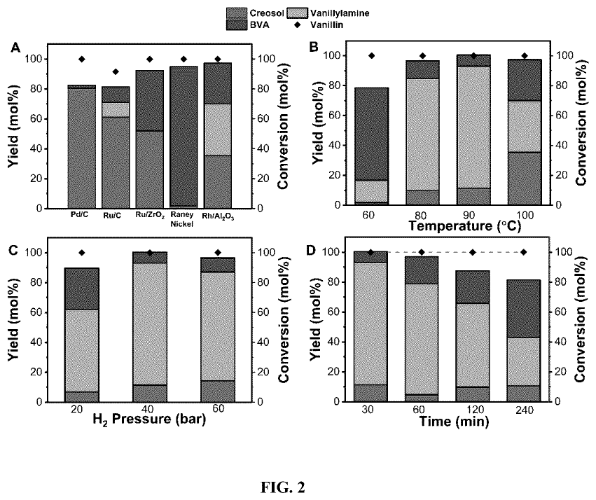

The metal catalyst and support strongly affected the selectivity of the reductive amination. The initial conditions and catalysts were adopted from recent literature. As shown in FIG. 2(a), amalgams, like Raney nickel catalysts, lead to the secondary amine (BVA) while no reduced products (creosol or the primary amine) formed. Strong reducing metals, such as Pd and Ru on carbon, produced exclusively creosol. Rh/Al₂O₃ exhibited the highest selectivity to the desired vanillylamine (a yield of 34.6%). Ru/C resulted in a vanillylamine yield of 10.4%.

The maximum vanillylamine yield was obtained at 40 bar H₂ in 30 minutes at a temperature of 90° C. Temperature profoundly affected selectivity of the reductive amination reaction (FIG. 2(b)). At a temperature of 100° C., the carbonyl group in vanillin was reduced, thereby producing creosol in high yield. Intriguingly, selectivity shifted to dimerized products below 80° C. The hydrogen pressure and reaction time were also optimized (FIG. 2(c) and FIG. 2(d)).

### Reductive Amination of Furfural

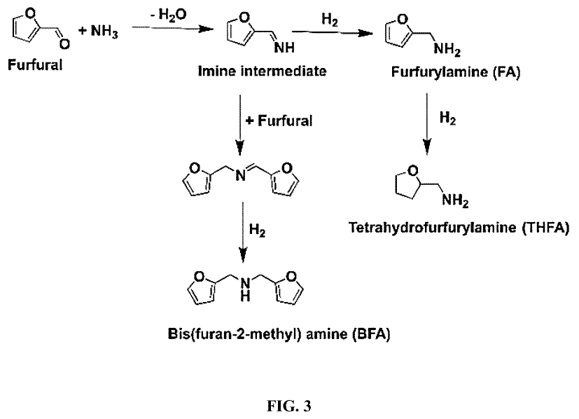

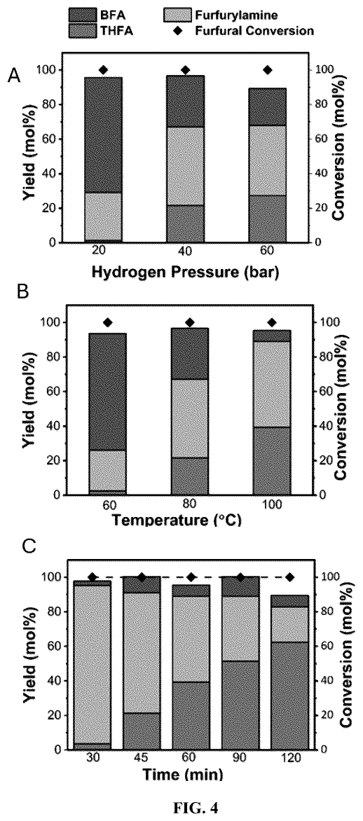

The reductive amination of furfural proceeded via the same mechanism shown in FIG. 3. A hydrogen pressure of 40 bar (FIG. 4(a)), a temperature of 100° C. (FIG. 4(b)), with a reaction time of 30 minutes, gave a 93% amine yield (FIG. 4(c)). Lower temperatures favored the secondary amine (60% and 30% selectivity at 60° C. and 80° C., respectively). Longer reaction times promoted ring-hydrogenation, consistent with prior work on ring hydrogenation of furfural and 5-hydroxymethyl furfural (HMF) over Ru and Rh catalysts.

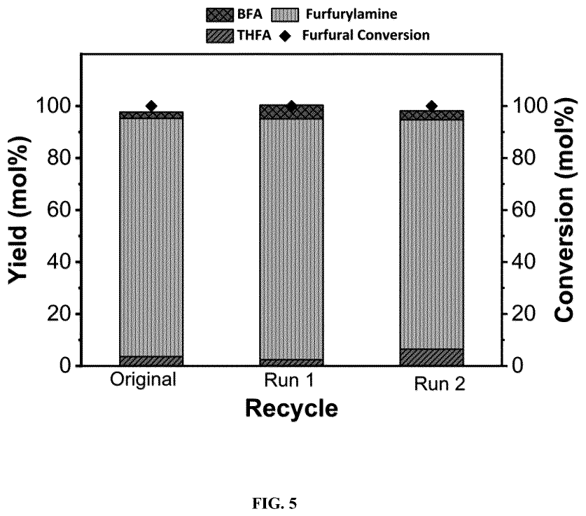

Upon completion of the reaction, the Rh/Al₂O₃ was recovered through vacuum filtration. The collected catalyst was washed multiple times with DI water and re-used for the subsequent reaction. The recyclability data, given in FIG. 5, indicates a slight change in product distribution but the overall product yield remained the same.

### Carbonylation of Furfurylamine

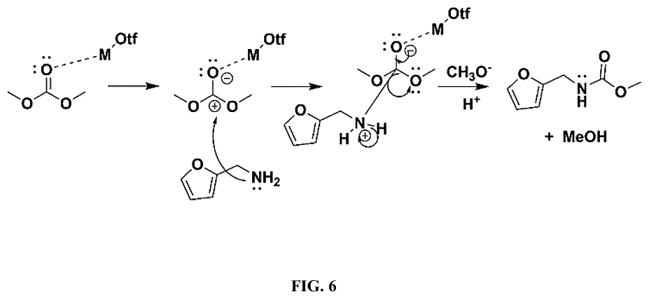

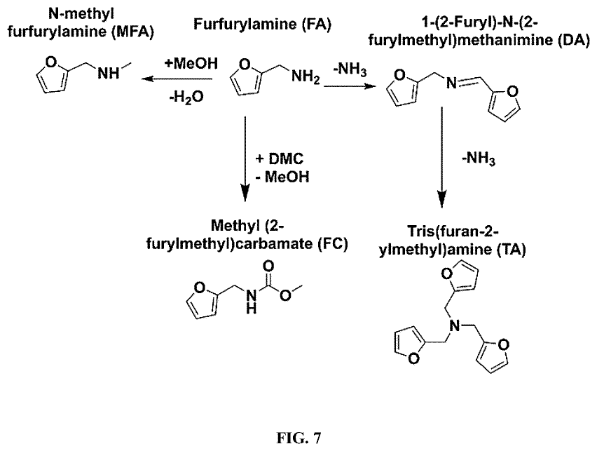

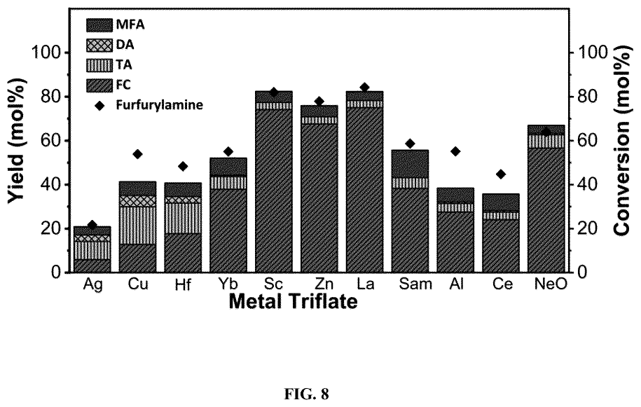

Carbonylation was performed on furfurylamine in the presence of various metal triflates. The mechanism involving dimethyl carbonate (DMC) activation has been extensively studied, and involves the coordination of the metal ion with the carbonyl oxygen to facilitate the attack of the amine group on the positively charged carbonyl carbon shown in FIG. 6. This leads to the formation of carbamate ester products and the generation of methanol as a stoichiometric byproduct FIG. 7. The reaction network for this carbonylation with furfurylamine is also given in FIG. 7. The activity of metal triflates correlates with their Lewis acidity, as shown in FIG. 8, with high conversions of furfurylamine to FC for La (82%), Sc (81%), and Zn (78%). Byproducts included tris(furan-2-ylmethyl)amine (TA), N-methyl furfurylamine (MFA), and the 1-(2-furyl)-N-(2-furylmethyl) methanimine (DA).

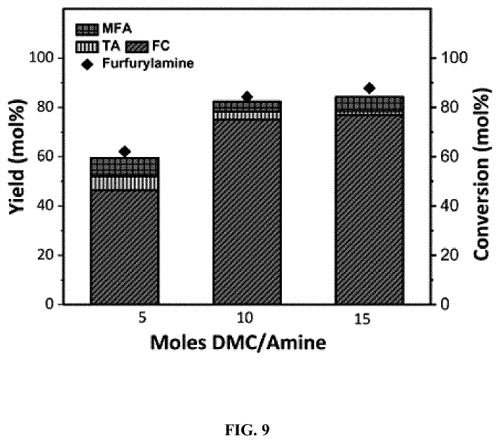

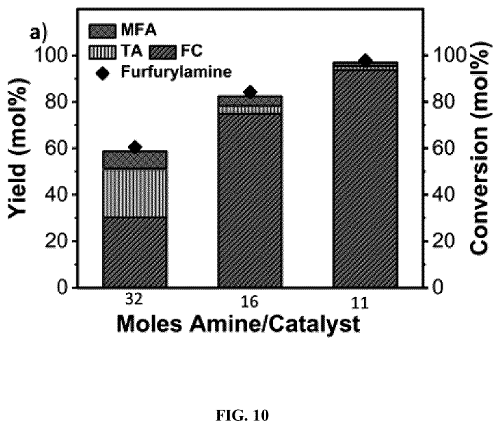

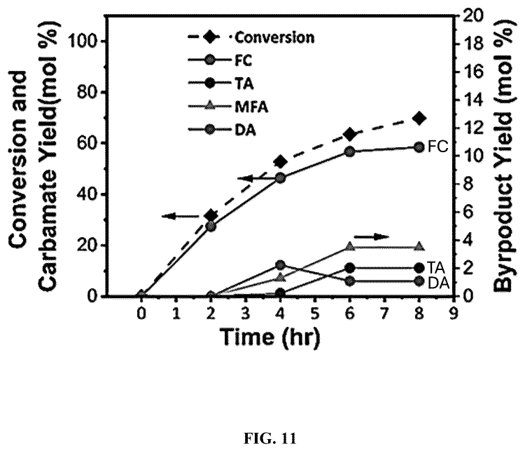

Optimization of the catalyst/amine ratio and the DMC/amine ratio was conducted with La(OTf)₃. Higher DMC/amine ratios increased the activity and selectivity to FC (FIG. 9). The conversion of furfurylamine increased from 61% to 82% when the DMC/amine ratio was doubled. A possible explanation for this increase is that excess DMC allowed for better mixing, thereby increasing conversion. Higher catalyst loadings increased furfurylamine conversion and selectivity to the primary carbamate (FIG. 10). The highest yield of FC (95%) was achieved at a DMC/amine/catalyst ratio of 10/1/0.1. Time-dependent studies (FIG. 11) provided insight into the mechanism of the carbonylation of furfurylamine, illustrated in FIG. 8. Methylation of furfurylamine was found to occur after significant amounts of FC and methanol were produced stoichiometrically (0% yield at 2 h, 3% yield at 8 hours). This indicates that methylation was due to the methanol generated in situ. Triflates can catalyze methylation by coordinating with amine groups. Coordination of the metal with the amine group and concomitant dehydrogenation of FA and DA could also lead to the formation of triamines. The decrease in the secondary imine yield at longer times (3% at 4 hours and 0.5% at 8 hours) and the concurrent increase in triamine yield (0 at 4 hours to 2% at 8 hours) also indicated that these byproducts are produced sequentially.

### Carbonylation of Vanillylamine

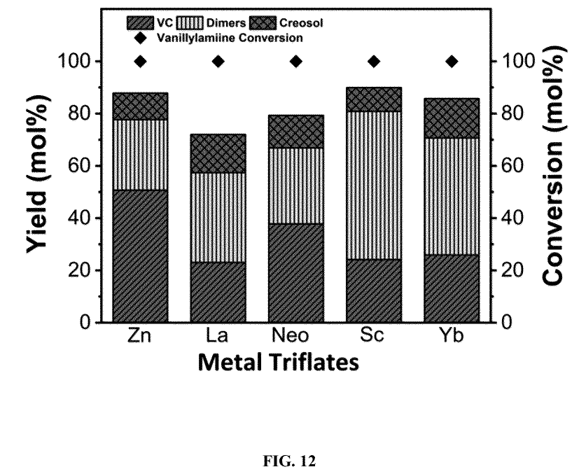

Carbonylation of vanillylamine gave a modest yield to methyl (4-hydroxy-3-methoxybenzyl) carbamate (VC) (52% shown in FIG. 12) possibly due to polymerization and dimerization of vanillylamine.

### Insecticidal Activity

The performance of the synthesized biobased insecticides (FC and VC) were compared to carbofuran, one of the most toxic synthetic carbamate insecticides in use (Table 1).

TABLE 1
Probit analysis results for adult Alphitobius diaperinus exposed
in vial bioassays to carbofuran (CF), methyl (2-furylmethyl)carbamate
(FC), and methyl (4-hydroxy-3-methoxybenzyl) carbamate (VC).

| | Pooled number of | | |
|---|---|---|---|
| Chemical | beetles for analysis | Slope (SE) | LC₅₀(95% CI) (ng/cm²) |
| CF | 524 | 0.49 (0.07) | 251,258 |
| | | | (106,755-560,330) |
| FC | 210 | 2.61 (0.45) | 254,226 |
| | | | (160,787-518,110) |
| VC | 375 | 5.25 (0.84) | 6,660,622 |
| | | | (5,806,798-9,961,100) |

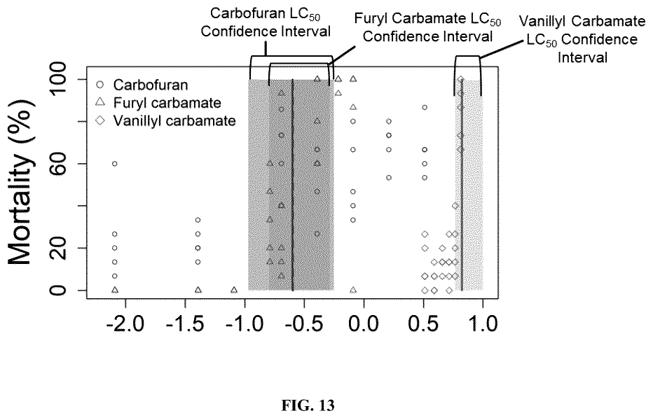

The median lethal concentration (LC₅₀) for FC was equivalent to the LC₅₀ for carbofuran, with confidence intervals broadly overlapping (FIG. 13). The LC₅₀ for VC was two orders of magnitude higher than those for carbofuran and FC, thereby indicating lower toxicity. The comparable performance of FC with one of the most toxic synthetic insecticides, on the other hand, is encouraging and signifies that grafting biomass derived monomers with molecular precision has the potential to replicate toxicity of commercial insecticides.

### Ecotoxicity Evaluation

A major environmental concern with the conventional pesticides is the potential to bioaccumulate and threaten the ecosystem. Carbofuran is highly toxic to birds, mammals, marine fish, and freshwater invertebrates on an acute basis. Other carbamates, such as aldicarb and carbaryl, threaten aquatic species such as snakehead fish and salmon, potentially killing thousands in affected water bodies. Carbamate insecticides are acetylcholinesterase (AChE) inhibitors (Mode of action group 1A) and, despite their potent insecticidal activity, are under stringent regulations due to their non-specificity.

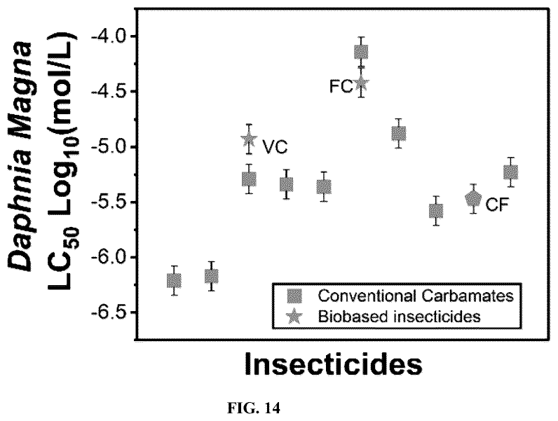

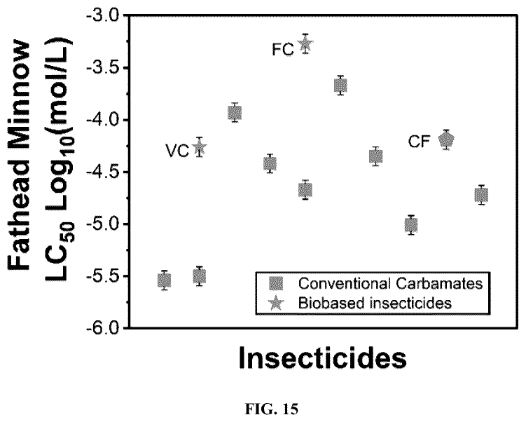

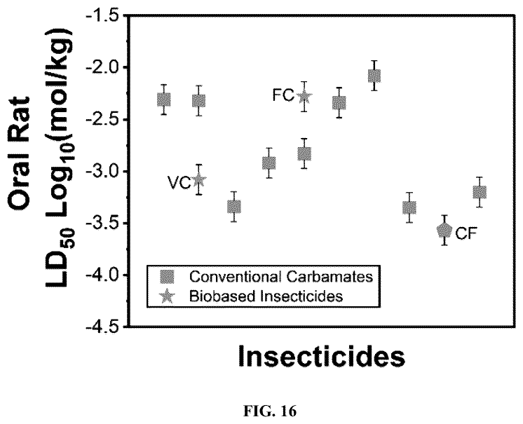

Therefore, examination of the biobased insecticides FC and VC was evaluated to determine their ecotoxicities. The aquatic and mammalian toxicity of FC and VC was predicted using the peer-reviewed TEST software and compared with the commercial carbamates (FIG. 14, FIG. 15 and FIG. 16). The raw toxicity data for three non-target species: rats (Rattus rattusa, a mammal), fathead minnows (Pimephales promelas, a freshwater fish), and Daphnia magna (a planktonic crustacean) is given in Table 2.

TABLE 2
Raw data of the ecological risks of insecticidal active ingredients simulated using TEST

| | | Fathead | Daphnia | | |
|---|---|---|---|---|---|
| | Oral Rat LD₅₀ | Minnow LC₅₀ | Magna LC₅₀ | | |
| Compounds | (log(mol/kg)) | (log(mol/L)) | ((log(mol/L)) | Log(Kow) | AMR |
| Phenmedipham | −2.31 | −5.54 | −6.21 | 3.42 | 80.29 |
| Desmedipham | −2.32 | −5.5 | −6.17 | 3.28 | 80.00 |
| Pirimicarb | −3.34 | −3.93 | −5.29 | 2.01 | 66.65 |
| Propoxur | −2.92 | −4.42 | −5.34 | 2.30 | 56.39 |
| 1-Naphthalenol, 1-(N- | −2.83 | −4.67 | −5.36 | 2.50 | 57.21 |
| methylcarbamate) | | | | | |
| Molinate | −2.34 | −3.67 | −4.14 | 2.50 | 53.82 |
| Carbendazim | −2.08 | −4.35 | −4.88 | 1.65 | 49.15 |
| Thiodicarb | −3.35 | −5.01 | −5.58 | 2.10 | 87.00 |
| Carbofuran | −3.57 | −4.19 | −5.47 | 2.23 | 59.59 |
| Methiocarb | −3.2 | −4.72 | −5.23 | 3.10 | 54.73 |
| Methyl (2- | −2.28 | −3.27 | −4.49 | 1.00 | 37.99 |
| furylmethyl)carbamate (FC) | | | | | |
| Methyl(4-hydroxy-3- | −3.08 | −4.26 | −4.93 | 1.32 | 53.76 |
| methoxybenzyl) carbamate | | | | | |
| (VC) | | | | | |
| Eugenol | −1.85 | −4.10 | −4.36 | 3.42 | 37.99 |
| Anthraquinone | −1.82 | −4.70 | −3.98 | 3.28 | 53.76 |
| 2-Phenylethyl propionate | −1.68 | −4.42 | −4.51 | 2.01 | 34.51 |
| 3-Phenylprop-2-enal | −1.81 | −4.77 | −3.98 | 2.30 | 63.99 |
| Indole-3-acetic acid | −2.16 | −4.21 | −4.21 | 2.50 | 42.21 |
| N-Benzyladenine | −1.97 | −4.14 | −4.37 | 2.50 | 48.45 |
| Estragole | −2.14 | −4.48 | −4.31 | 1.65 | 51.41 |
| 2-(5-Phenylmethoxypent-1- | −2.32 | −5.34 | −5.54 | 2.10 | 48.50 |
| en-2-yl)aniline | | | | | |
| Salicylic Acid | −2.32 | −3.15 | −3.15 | 2.23 | 61.15 |

Compared to the available experimental toxicity data for all three species, it was discovered that at the log scale, TEST has a mean percentage error below 15% (Table 3), indicating its accuracy for the subset of compounds selected.

TABLE 3
Mean percentage error of TEST for the compounds screened.

| | | Fathead Minnow | Daphnia Magna |
|---|---|---|---|
| Species | Oral Rat LD₅₀ | LC₅₀ | LC₅₀ |
| Mean Percentage | 14.4 | 9.1 | 13.3 |
| Error | | | |

FC has one of the highest LD₅₀ for rats and the highest LC₅₀ for fathead minnows indicating that it is less toxic than conventional carbamates. Carbofuran's (CF) ecotoxicity is an order of magnitude higher than FC in all three categories despite having similar toxicity toward the target pest. EPA's classification for pesticide toxicity to humans and aquatic species ranges from acutely toxic (category I) to non-toxic (category IV). FC's predicted oral median lethal dose for rats, 1160 mg/kg, and LC₅₀ of 83 mg/L place it in toxicity category III (500-5000 mg/kg oral and 10-100 mg/L fathead minnow). This corresponds to a slightly toxic label, while most conventional pesticides carry acute toxicity designations (category I). These results indicate that FC is an ecologically safer insecticide than many commercial pesticides currently on the market. VC has a predicted oral LD₅₀ of 175 mg/kg and LC₅₀ of (11 mg/L) placing it in category II (moderately toxic) for mammals and borderline category Ill for fish. These results indicate that FC is a potent but also less ecologically toxic insecticide and signify the sustainable impact these biomass-derived compounds can bring to crop protection methods.

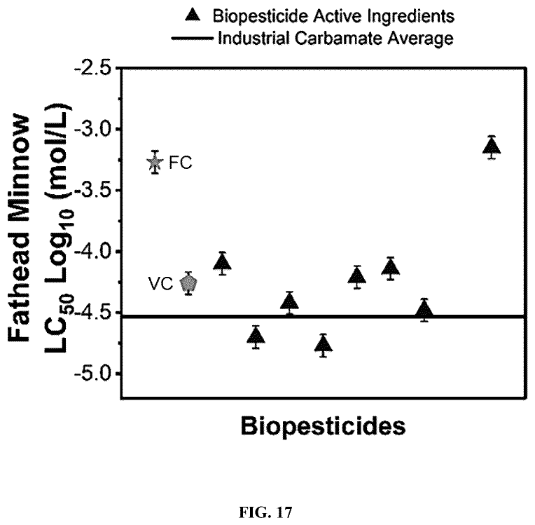

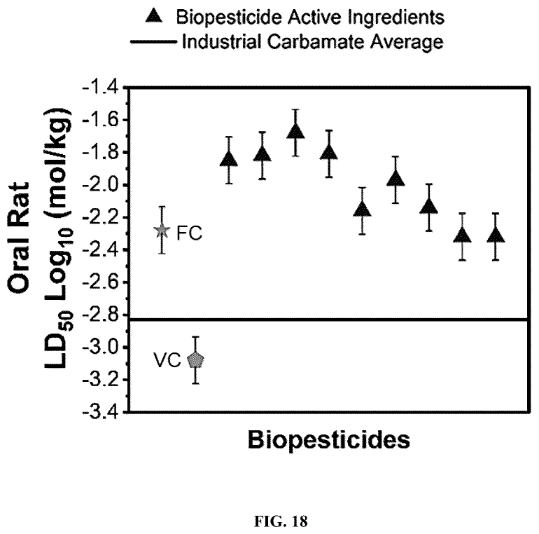

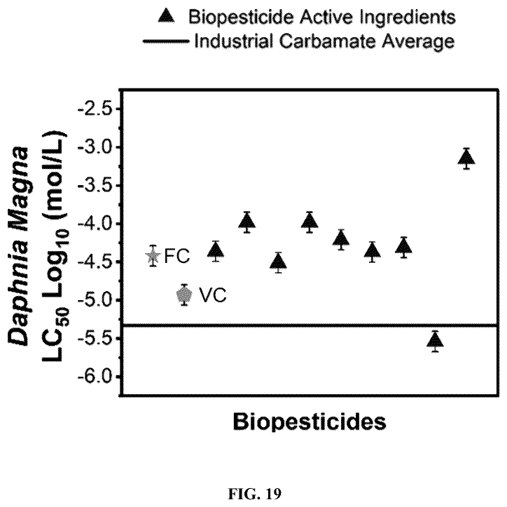

Next, the ecotoxicity of the biobased insecticides prepared according to the Example (FC and VC) were compared to several biopesticide active ingredients with aromatic and heterocyclic groups that are registered with the EPA. FC displayed lower toxicity towards fish (FIG. 17), higher toxicity towards mammals (FIG. 18) and comparable toxicity towards Daphnia magna (FIG. 19). VC displayed similarly high toxicity to the commercial carbamates. The higher toxicity towards mammals could be due to the carbamate functional group. However, despite the presence of the carbamate functionality, FC emerges as one of the less toxic compounds for fathead minnows.

Specific functional groups can impact the toxicity of pesticide end-life products. Incorporating electron-donating groups, such as ether or amines, is particularly advantageous to reduce the molecule's lipophilicity and enhance its potential for photodegradation. Specifically, substituting the nitro groups (electron withdrawing) with amines (electron donating) on 2-methyl-4,6-dinitrophenol (DNP) remarkably increased its photodegradation potential and decreased its bioavailability, shifting it to a lower risk chemical. The results of the studies conducted in this Example suggest that similar ecological benefits can occur by substituting unreactive benzene cores found in many commercial pesticides with an electron-dense, five-membered ring containing an oxygen heteroatom.

FC's low bioavailability, attributed to a low octanol water partition coefficient (K_{ow}), counteracted the potential toxic effects of the carbamate group in fish. It is worth noting that bioavailability is not deemed significant in the toxicity simulations for mammals, as the model focuses on the oral dosage of the active ingredient rather than the diffusion of the compound into nearby organisms.

The Ghose-Crippen octanol/water partition coefficient (K_{ow}) was predicted using TEST. K_{ow} is a useful index for quantifying insecticides aquatic toxicity since it indicates whether the compound endures in the aqueous phase, such as the water body into which it is introduced, or infiltrates into organisms. A lower K_{ow} implies a reduced likelihood of a compound diffusing into aquatic species, and thus a reduced ecotoxicity. Conventional insecticides derived from benzene-based materials have a higher K_{ow}, the majority of them have log(K_{ow}) values >2. FC, on the other hand, has a K_{ow} of 1, representing an order of magnitude difference. The increased dipole moment and electron density in furans, compared with benzene rings, due to the oxygen heteroatom, resulted in a lower K_{ow} than commercial pesticides, thereby indicating a lower aquatic toxicity. Consequently, the favorable electronic properties of the furan ring are crucial contributors to the low ecotoxicity of FC. Another important molecular descriptor for ecotoxicity is a compound's molar refractivity. We thus also predicted the Ghose Crippen molar refractivity (AMR) for the different compounds, shown in Table 2. A high AMR correlates to higher ecotoxicity.

To corroborate the findings from TEST, the aquatic toxicity of VC and FC to fish and green algae was evaluated using ECOSAR (Ecological Structure Activity Relationships). The tabulated values are shown in Table 4.

TABLE 4
Tabulated ECOSAR LC₅₀ predictions
with the appropriate chemical class

| | | Fathead | |
|---|---|---|---|
| | | Minnpw | Green |
| | | Predicted | Algae |
| | | LC₅₀ | EC₅₀ |
| Compounds | ECOSAR Class | (mg/L) | (mg/L) |
| Phenmedipham | Carbamate Esters | 4.92 | 0.18 |
| Desmedipham | Carbamate Esters | 5.80 | 0.19 |
| Pirimicarb | Carbamate Esters | 38.30 | 0.31 |
| Propoxur | Carbamate Esters | 33.94 | 0.27 |
| 1-Naphthalenol, 1-(N- | Carbamate Esters, Phenyl | 3.73 | 2.23 |
| methylcarbamate) | | | |
| Molinate | Thiocarbamates, Mono | 14.23 | 0.43 |
| Carbendazim | Carbamate Esters | 56.45 | 0.30 |
| Thiodicarb | Oxime Carbamate Ester | 1.22 | 0.48 |
| Carbofuran | Carbamate Esters, Phenyl | 4.27 | 2.59 |
| Methiocarb | Carbamate Esters | 3.83 | 0.14 |
| Methyl (2- | Carbamate Esters | 109.64 | 0.95 |
| furylmethyl)carbamate | | | |
| (FC) | | | |
| Methyl(4-hydroxy-3- | Phenols | 155.65 | 0.45 |
| methoxybenzyl) | | | |
| carbamate (VC) | | | |
| Eugenol | Phenols | 7.94 | 14.18 |
| Anthraquinone | Quinones | 0.05 | 0.05 |
| 2-Phenylethyl | Esters | 2.77 | 1.60 |
| propionate | | | |
| 3-Phenylprop-2-enal | Vinyl/Allyl Aldehydes | 0.18 | 23.55 |
| Indole-3-acetic acid | Pyrazoles/Pyrroles -acid | 110.04 | 150.22 |
| N-Benzyladenine | Neutral Organics | 154.26 | 68.02 |
| Estragole | Neutral Organics | 5.86 | 4.97 |
| 2-(5-Phenylmethoxypent- | Aliphatic Amines | 0.53 | 0.04 |
| 1-en-2-yl)aniline | | | |
| Salicylic Acid | Vinyl/Allyl Alcohols- | 38.33 | 835.51 |
| | acid | | |

FC (109.6 mg/L) and VC (112.5 mg/L) are shown to have higher LC₅₀'s for fish compared to all the industrial carbamates and all but two biopesticides. For green algae, FC (0.95 mg/L) and VC (0.45 mg/L) were safer than the majority of industrial carbamates. Interestingly, all the carbamate molecules had low (<2.5 mg/ml) EC₅₀ (Effective Concentration) values, while the majority of biopesticides were safer (>10 mg/ml), indicating that green algae is particularly sensitive to the carbamate functional groups.

These results clearly distinguish FC from conventional carbamate insecticides and show that its risk to aquatic environments is low, even when compared with the inherently safer biopesticides. These results also indicate that similar biomass-based insecticides can alleviate current ecotoxicity concerns associated with the carbamate class of insecticides.

### Extending the Synthetic Scope

The array of bio-derived furans, diols, and aromatics that can be transformed into amines underscores the broad scope of the synthesis method used in this Example and those disclosed herein. To showcase this versatility, six carbamate molecules have been synthesized from other biomass-derived monomers. Table 5 details their structures, along with the achieved yield.

TABLE 5
Different carbamates synthesized using the methodology of Example 1.

| | | | Amine | |
|---|---|---|---|---|
| | | | Conversion | Carbamate |
| Compound | Acronym | Structure | (mol %) | Yield (mol %) |
| Methyl (tetrahydro-2- furanylmethyl)carbamate | THFC | 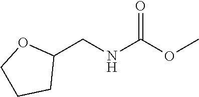 | 100 | 91 |
| Methyl (6- hydroxyhexyl)carbamate | HOHC | 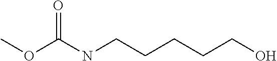 | 100 | 87 |
| Methyl (4- hydroxybenzyl)carbamate | PC* | 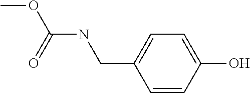 | 100 | 32 |
| Methyl (2- thienylmethyl)carbamate | TC | 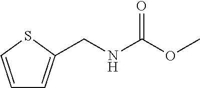 | 86 | 82 |
| Methyl [(5-methyl-2- furyl)methyl]carbamate | MFC | 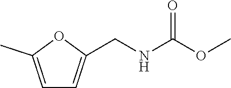 | 97 | 88 |
| Methyl (3,4,5- trimethoxybenzyl)carbamate | MTBC | 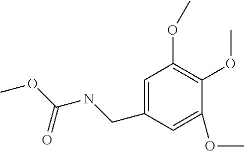 | 100 | 67 |

The TC monomer was less reactive than furan-based amines with similar high (>95%) conversions. PC (32% carbamate yield) was more susceptible to dimerization than VC (52% carbamate yield) and MTBC (67% carbamate yield), resulting in lower yields of the carbamate (36%). The ecotoxicity predictions of these new compounds are shown in Table 6.

TABLE 6
Ecotoxicity predictions for the different
biobased carbamates synthesized

| | Fathead | | |
|---|---|---|---|
| | Minnow | | |
| Compounds | LC₅₀(mol/L) | AMR | Log(Kow) |
| Methyl (tetrahydro-2- | 162.70 | 39.24 | 0.38 |
| furanylmethyl)carbamate (THFC) | | | |
| Methyl (6-hydroxyhexyl)carbamate | 44.22 | 45.99 | 1.03 |
| (HOHC) | | | |
| Methyl (4-hydroxybenzyl)carbamate | 30.75 | 47.29 | 1.33 |
| (PC) | | | |
| Methyl (2-thienylmethyl)carbamate | 46.00 | 44.43 | 1.55 |
| (TC) | | | |
| Methyl [(5-methyl-2- | 56.67 | 42.83 | 1.14 |
| furyl)methyl]carbamate (MFC) | | | |
| Methyl (3,4,5- | 16.81 | 64.99 | 1.55 |
| trimethoxybenzyl)carbamate (MTBC) | | | |

Structural modifications to the furan-based compounds THFC, MFC, and TC influenced the LC₅₀ toward fathead minnows. THFC showed an unprecedented lack of toxicity toward fathead minnows (162 mg/L=category IV), whereas MFC and TC were more ecotoxic (56 mg/L and 46 mg/L, respectively). The structural modifications significantly impacted K_{ow}, as shown in Table 6. MFC and TC had higher K_{ow} values and, interestingly, THFC had a K_{ow} lower than FC by close to a factor of 3. Adding a methyl chain to the furan ring decreased the water solubility of MFC and substituting the oxygen heteroatom with sulfur reduced the polarity as sulfur is less electronegative than oxygen. The solubility of THFC can be explained by the localization of the lone pair on the oxygen heteroatom. The resonance of the unsaturated furan ring delocalizes the negative charge, reducing the overall polarity of the molecule.

PC and MTBC, the structural analogues of VC, exhibited an interesting trend in predicted ecotoxicity. According to the predictions, PC is safer since its LC₅₀ and LD₅₀ to fish are thrice that of VC (30 mg/L vs 11 mg/L and 524 mg/kg vs 175 mg/kg). These predictions change the category of this active ingredient to category III (524 mg/kg) for mammals and firmly in category III (30 mg/L) for fish. Methoxy and alcohol groups are not predicted to significantly impact log(K_{ow}) but could increase AMR. The addition of methoxy groups can clearly increase the AMR for VC and MTBC, resulting in higher ecotoxicities.

These observations are significant as they underscore favorable ecological property engineering by tuning the substituents and heteroatom of the furan and phenolic ring to impact K_{ow} and the AMR of pesticide molecules.

### Ecotoxicity Validation by Honeybee Toxicity Measurements

Due to the limitations of validating the ecotoxicity experimentally on aquatic and other species given in TEST predictions, honeybee toxicity measurements are considered as an index to determine ecotoxicity of an insecticide. The honeybee toxicity studies were conducted by a third-party company, Eurofins EAG Agroscience, LLC, as a standard measure of safety for pollinators. The experimental methods involve exposing worker honeybees to different doses of chemical dissolved in acetone, then keeping them in ideal temperature/humidity/light conditions with unlimited access to food, and measuring mortality after two days. Water and acetone (the solvent) were included as negative controls, and the highly toxic organophosphate dimethoate was included as a positive control. Promisingly, the 24-hr LD₅₀ of our compound was 126 μg/bee (and 48-hr LD₅₀=75 μg/bee), which is orders of magnitude less toxic than positive control, carbamate or neonicotinoid insecticides, and on par with newer diamide insecticides that are considered generally safe for honeybees and other pollinators.

### Fate of Degradation Products

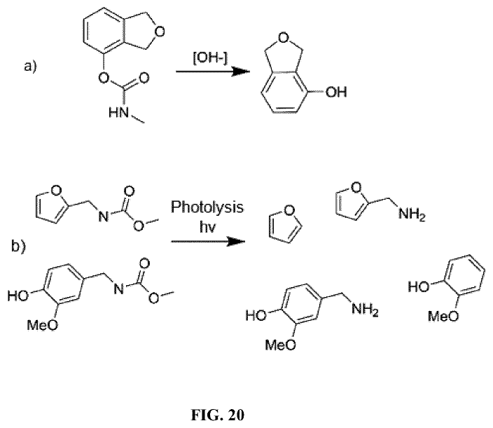

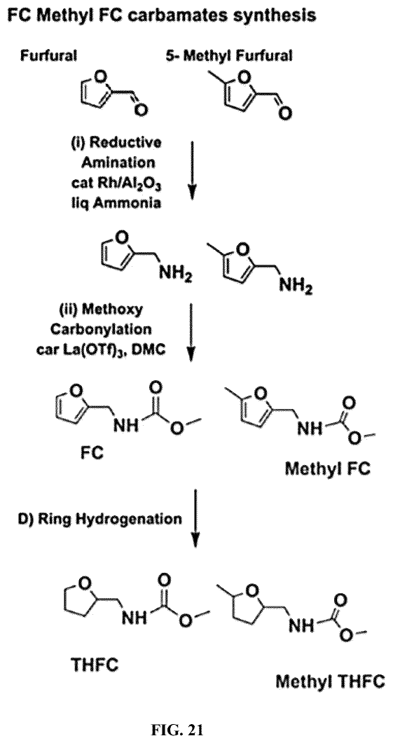

The fate of the end-life environmental degradation products of FC and VC was next evaluated since the end-life products of industrial carbamates can often be harmful. As shown in FIG. 20, the carbofuran undergoes ester group hydrolysis, resulting in an alkyl amine, CO₂, and an organic residue. To evaluate the degradation of VC, FC and carbofuran, the alkaline hydrolysis was conducted using different pH buffers, and the metabolites were extracted with ethyl acetate and analyzed. Carbofuran completely hydrolyzed to phenol hydro furan after 12 hours in a buffer of pH 10 (FIG. 21(a)). Surprisingly, FC remained stable and persisted even at a higher pH of 12. This unexpected stability could be attributed to the orientation of the carbamate group. That is, the ester linkage in carbofuran is connected to the aromatic ring, thereby making the ester groups highly susceptible to hydrolysis. In contrast, the —NH group in FC and VC is connected to the furan or aromatic ring, thereby providing hydrolysis resistance. Due to their respective amines or aromatic moieties (FIG. 21(b)), it is speculated that FC and VC degrade in a manner similar to Fenoxycarb, a carbamate insecticide that mimics a juvenile hormone (Group 7) and is used to disrupt insect development. Fenoxycarb's carbamate group has a similar orientation to FC and VC, and Fenoxycarb has been shown to degrade via photolysis. The corresponding amine for FC and VC was confirmed as the main degradation product using biotransformer. The degradation products were also assessed with TEST as shown in Table 7.

TABLE 7
Ecotoxicity predictions for the degradation products of FC and VC.

| | Oral | Fathead | Daphnia |
|---|---|---|---|
| | Rat LD₅₀ | Minnow LC₅₀ | Magna LC50 |
| Compounds | (log(mg/kg)) | (log(mg/L)) | ((log(mg/L)) |
| Furfurylamine | 207.57 | 227.91 | 39.42 |
| Vanillylamine | 397.83 | 111.18 | 9.49 |

The respective amines were predicted to be less toxic to Daphnia magna and the fathead minnow. Furfurylamine was also predicted to be more toxic toward mammals than FC.

### Technoeconomic Analysis

Using Aspen 12's economic analyzer, the technoeconomics of FC were evaluated. The plant's capacity was assumed to be 1000 tons/yr, approximately 20% of worldwide carbofuran production. The breakdown of capital and operating costs, along with the costs of each component needed to produce FC, are provided in Tables 8 and 9.

TABLE 8
Breakdown of capital and operating costs.

| Capital Cost(USD$) | | Operating Cost(USD$/year) | |
|---|---|---|---|
| Purchased Equipment | 2,660,200.20 | Utilities | 157009.4 |
| Equipment Setting | 52,042.40 | Operating Labor | 920000 |
| | | Cost | |
| Piping | 317,811.40 | Maintenance Cost | 89500 |
| Civil | 100,855.40 | Operating Charges | 230000 |
| Steel | 85,450.00 | Plant Overhead | 504750 |
| Instrumentation | 924,078.40 | G and A Cost | 212639 |
| Electrical | 1,779,744.00 | Raw Materials | 756727.9 |
| Insulation | 72,916.40 | | |
| Paint | 32,217.10 | | |
| Other | 3,176,400.20 | | |
| Subcontracts | 0 | | |
| G and A Overheads | 228,555.50 | | |
| Contract Fee | 396,758.30 | | |
| Escalation | 0 | | |
| Contingencies | 1,768,865.40 | | |
| Special Charges | 0 | | |
| Total Project Cost | 11,595,894.70 | | |
| Adjusted Total | 11,455,724.54 | | |
| Project Cost | | | |
| Miscellaneous | 3,519,881.50 | | |

TABLE 9
Cost per component

| Stream | Cost (USD$/kg) |
|---|---|
| Citric acid | 1.35 |
| Dimethyl Carbonate | 0.62 |
| Furfural | 1.00 |
| Water | 0.05 |
| Hydrogen | 1.57 |
| Ammonia | 0.94 |
| Dichloromethane | 0.87 |

A minimum selling price (MSP) of the active ingredient is predicted at 11.14 $/kg, a third of carbofuran's price of 34 $/kg, and half of carbaryl's selling price of 23 $/kg. This cost advantage can be attributed to using inexpensive commodity chemicals, including furfural, ammonia, and dimethyl carbonate, for synthesizing a high-value active ingredient. Furthermore, the process involves only two synthesis steps and easy purification methods, while conventional carbaryl and carbofuran synthesis involves four to five steps with a cost associated with separation in each step.

### Principles of Green Chemistry

Table 10 highlights the 8 green chemistry principles that were satisfied in the Example.

TABLE 10
Principles of Green Chemistry

| | |
|---|---|
| 1. | Prevention |
| 2. | Atom Economy |
| 3. | Less Hazardous Chemical Syntheses |
| 4. | Designing Safer Chemicals |
| 5. | Safer Solvents and Auxiliaries |
| 6. | Design for Energy Efficient |
| 7. | Use of Renewable Feedstocks |
| 8. | Reduce Derivatives |
| 9. | Catalysis |
| 10. | Design for Degradation |
| 11. | Real-time Analysis for Pollution Prevention |
| 12. | Inherently Safer Chemistry for Accident Prevention |

The synthesis method used to produce FC demonstrated a high atom economy of 74%, meeting criterion 2. The ecotoxicity analysis data shown in Tables 2 and 4 satisfy criterion 3. The ecotoxicity of the reagents utilized in the synthesis of this Example, such as dimethyl carbonate and furfurylamine, and the intermediates of carbofuran, such as 2,2 dimethyl-7-hydroxycoumaran and methyl isocyanate, are shown in Table 11.

TABLE 11
Ecotoxicity predictions for the reagents used
in the synthesis of FC and carbofuran

| | | Fathead | Daphnia |
|---|---|---|---|
| | Oral Rat LD₅₀ | Minnow LC₅₀ | Magna LC₅₀ |
| Compounds | (log(mg/kg)) | (log(mg/L)) | ((log(mg/L)) |
| Furfurylamine | 207.57 | 227.91 | 39.42 |
| Dimethyl carbonate | 8644.20 | 600.86 | 167.35 |
| Methyl isocyanate | 155.58 | 88.32 | 34.73 |
| 2,2 dimethyl-7- | 503.11 | 43.86 | 8.54 |
| hydroxycoumaran | | | |

The latter reagents have lower LD₅₀ and LC₅₀ values to mammals and aquatic life. This demonstrates that the synthesis in this Example utilizes safer chemicals and solvents, satisfying criteria 4 and 5. Furfural is a biomass-derived platform chemical, meeting criterion 7. The synthesis also utilizes recyclable catalysts and includes no protection groups, satisfying criteria 8 and 9. Finally, methyl isocyanate is a highly volatile chemical, responsible for the worst industrial chemical disasters. Eliminating the use of methyl isocyanate in the synthesis achieves the goal of inherently safer chemistry for accident prevention.

### III. Conclusion of Example 1

Sustainably sourced pesticides are needed to reduce reliance on synthetic pesticides derived from fossil fuels at an annual volume of 5.6 billion pounds. Emerging biopesticides are projected to be the future of crop protection but are limited by short supply. In this context, Example 1 introduced a synthesis of structurally similar insecticidal active ingredients with molecular precision from biomass resources. This synthesis combines two well-established industrial chemistries, reductive amination and carbonylation, to upgrade commercially available biobased platform molecules, furfural, and vanillin, to carbamate functionalized insecticides, FC and VC, satisfying 8 out of 12 criteria in green chemistry synthesis. The conditions were optimized to achieve ˜95% yield of furfurylamine and FC in the reductive amination and carbonylation steps. The insecticidal activity evaluation against a known pest, lesser mealworm beetles (Alphitobius diaperinus), showed that FC has comparable potency to the widely used and highly toxic commercial insecticide, carbofuran. VC was less effective and can be a potential candidate as herbicide or plant growth regulator. Ecotoxicity predictions using the EPA developed software TEST revealed that the synthesized biobased compounds are in ecotoxicity category III (minimal ecotoxicity) and thus considerably safer than most conventional pesticides. The predictions also show that FC is safer than most biopesticide active ingredients toward fathead minnows' aquatic species. The honeybee toxicity measurements show that FC is orders of magnitude less toxic than carbamate or neonicotinoid insecticides, and on par with newer diamide insecticides that are considered generally safe for honey bees and other pollinators. Octanol and water partition coefficient predictions indicate that these biobased insecticides have lower potential for bioaccumulation in aquatic species. The lower ecotoxicity for FC can be attributed to the electronic properties of its furan ring. By modifying the ring substituents, the ecotoxicity of these biobased insecticides can be tuned. Finally, technoeconomic analysis showcases the economic viability for producing FC. Overall, the data gathered from performing the experiments of this Example provide a foundation for biomass-derived, environmentally safer pesticides that could be transformative for crop protection.

### Example 2: Combinatorial Design of Bio-Based Carbamate Functionalized Insecticide

This example details the generation of ten insecticide compounds that were synthesized and evaluated against Alphitobius diaperinus.

### Materials and Methods of Example 2

Materials Anhydrous dimethyl carbonate and liquid ammonia were used as received from Sigma-Aldrich. Supported metal oxides and metal triflate catalysts were all purchased from Sigma-Aldrich. Furfural, hydroxymethyl furfural, furfurylamine. 5-methyl furfurylamine, methyl iodide, potassium tert-butoxide, diisopropyl ethylamine (DIPEA), methyl chloroformate, 5-chloromethyl furoate, 12 M HCl were all used as received from Sigma-Aldrich. 5-hydroxymethyl furfurylamine was purchased from Enamine. Anhydrous sodium sulfate, deuterated solvents d₆-DMSO, CDCl₃, and all hydrous solvents were purchased from Fischer. Metal oxides were reduced at 300° C. for 3 h at a ramp rate of 10° C./min under 100 cm³/min 10% hydrogen/helium flow.

### Reductive Amination

The reductive amination reactions were conducted in 50 mL Parr reactors. The aldehyde reactants, liquid ammonia, and the catalyst (Rh/Al₂O₃) were placed in a glass liner with a magnetic stir bar. After loading the glass liner in the Parr reactor, the reactor was closed, purged three times with nitrogen and twice with hydrogen, and then pressurized to the desired hydrogen pressure. After a designated reaction time, the reactor was cooled in an ice bath, depressurized, and opened. The products and remaining reactants were identified and quantified using a gas chromatogram mass spectrometer (GC-MS) equipped with a DB-5 column. Calibration curves were prepared for the amines quantified. The amines were extracted from the aqueous phase with 3 washes of dichloromethane (3×20 mL). The pH of the aqueous phase was reduced to about 10 using 1 M HCl for the extraction of HMFA.

### Aminolysis

The amines were mixed with 1.2 molar equivalents of DIPEA in a 50 mL round-bottom flask containing 25 mL of dichloromethane. The mixture was then placed in an ice bath and cooled to approximately 5° C. Meanwhile, 1.2 molar equivalents of the other reagent, methyl chloroformate, were mixed with 5 mL of methylene chloride in a 20 mL scintillation vial. This mixture was added dropwise to the amine solution. After 30 min, the reaction was quenched with 25 mL of DI water and the organic phase was collected. The catalyst DIPEA was removed from the product mixture by washing the organic phase with 2 aliquots of 20 mL 1 M HCl solution. The extraction procedure was modified for better extraction of the polar Methyl N-{[5-(hydroxymethyl) furan-2-yl]methyl}carbamate (HMFC). Initially, 60-70% of the carbamate was partitioned into the aqueous phase after the reaction was quenched. For better recovery, the aqueous phase was washed with 3×20 mL aliquots of ethyl acetate to recover about 90% of the desired carbamate. The combined organic extracts were then washed with brine, dried over sodium sulfate, and concentrated in vacuum to obtain the carbamate.

### Carbamate Product Quantification

Amines were quantified using calibration curves with standards purchased from Enamine and Sigma-Aldrich. A calibration curve was prepared for furfuryl carbamate. Based on this calibration, a response factor was calculated and applied to other synthesized carbamates according to the ECN for each molecule. The products were characterized by GCMS, liquid chromatography-mass spectrometry (LC-MS), electron spray ionization mass spectrometry (ESI-MS) and 1H and 13CNMR spectroscopy (Bruker AV400, d₆-DMSO, CDCl₃ solvent).

### Williamson Etherification

A strong base (potassium tert-butoxide) was first added to HMFC dissolved in 20 mL of THF and mixed for 30 min. Then, 1.2 molar equivalents of methyl iodide were added and the reaction continued for 6 hours. After quenching the reaction with DI water, the ether product was extracted with diethyl ether (3×20 mL). The combined organic extracts were washed twice with 10 mL of 2 M HCl followed with brine, dried over sodium sulfate, and concentrated using a rotary evaporator to obtain the desired ether carbamate product.

### Azidation

Methyl 5-(chloromethyl)-2-furoate (4 mmol) and 1.5 molar equivalents of NaN₃ were dissolved in MeOH/H₂O (18/2 mL). The mixture was then heated at 65° C. for 2 hours. The reaction mixture was then concentrated and recovered in diethyl ether (30 mL). The organic layer was washed with brine solution (20 mL), and then dried over sodium sulfate. Solvent removal under vacuum gave a whitish oil. The crude material was dissolved in MeOH (10 mL), and concentrated HCl (0.45 mL) and 10% Pd/C (0.05 g) were added at room temperature. The mixture was placed under 10 bar hydrogen pressure for 5 hours. Then, the catalyst was removed by filtration, and the solvent was removed under reduced pressure to yield yellow hydrochloride salt. The salt was washed repeatedly with diethyl ether to remove organic impurities and dried under a vacuum to give the desired product. The product yield was determined gravimetrically.

### Base Hydrolysis

To a solution of methyl 5-{[(methoxycarbonyl)amino]methyl}furan-2-carboxylate (ETMFC) (1 mmol) in THF (4 mL) and water (1 mL), LiOH (1.07 mmol) was added. The reaction mixture was stirred at 50° C. for 1 hour in a sealed vial and then cooled to room temperature. The mixture was diluted with 10 ml of water and then brought to a pH of 2 with 1 M HCl. The aqueous layer was extracted with ethyl acetate (3×20 mL). The combined organic extracts were washed with brine, dried over sodium sulfate, and concentrated in vacuum to obtain the carboxylic acid carbamate.

### Insecticide Bioassays

The median lethal concentration (LC₅₀) of chemicals was measured in adult lesser mealworm beetles (Alphitobius diaperinus) through vial bioassays. The beetles were sourced from a UD Agricultural Entomology Lab colony collected from a Delaware broiler house and housed in a controlled growth chamber (kept at 25° C., about 60% humidity, and in darkness). The colony was sustained on chicken layer pellets, supplemented with apple and carrot, and with Styrofoam blocks provided as pupation substrate. Preliminary screenings determined lethal concentration ranges for each chemical, achieved in glass vials by evaporating acetone-based insecticide dilutions. Five replicates per concentration (5-7 concentrations per chemical) and acetone-only controls were then set up for each chemical. Mortality and morbidity assessments were conducted on 15 beetles per vial after 7-day exposures. Statistical analysis involved probit regression, excluding vials displaying 0% mortality. Adjustments for heterogeneity factors were made based on chi-square goodness-of-fit test results. When inclusion of heterogeneity factors precluded estimates of fiducial limits (as was the case for HMFC OMe, FC, Methyl FC, and ETMFC) heterogeneity factors were not used.

### Experiments and Results of Example 2

### Synthetic Pathway Development and Application

Following structure selection, synthetic pathways were experimentally devised to make predicted furan and THE carbamates. Four building blocks were identified: Furfural, 5-hydroxymethylfurfural, 5-methyl furfural, and methyl 5-(chloromethyl)-2-furoate based on chemistries available to incorporate the carbamate functional group. The synthesis pathways are shown in FIGS. 21-23. NMR, mass spectroscopy, and LCMS were used to confirm the structure of the carbamates with the exception of HMFC and HMFC OMe, which were done via GC-MS. The molecules synthesized and their acronyms are presented in Table 12.

TABLE 12
Molecules Synthesized and Their Acronym

| Compound IUPAC name | Acronym |
|---|---|
| methyl N-[(furan-2-yl)methyl]carbamate | FC |
| methyl N-[(oxolan-2-yl)methyl]carbamate | THFC |
| methyl N-{[5-(hydroxymethyl)furan-2-yl]methyl} | HMFC |
| carbamate | |
| methyl N-{[5-(hydroxymethyl)oxolan-2-yl]methyl} | THMFC |
| carbamate | |
| methyl N-{[5-(methoxymethyl)furan-2-yl]methyl} | HMFC OMe |
| carbamate | |
| methyl N-{[5-(methoxymethyl)oxolan-2-yl]methyl} | THMFC OMe |
| carbamate | |
| 5-{[(methoxycarbonyl)amino]methyl}furan-2-carboxylic | FC COOH |
| acid | |
| 5-{[(methoxycarbonyl)amino]methyl}oxolane-2- | THFC COOH |
| carboxylic acid | |
| methyl N-[(5-methylfuran-2-yl)methyl]carbamate | Methyl FC |
| methyl N-[(5-methyloxolan-2-yl)methyl]carbamate | Methyl THFC |

### FC and Methyl FC Carbamates Synthesis: Reductive Amination and Methoxy Carbonylation

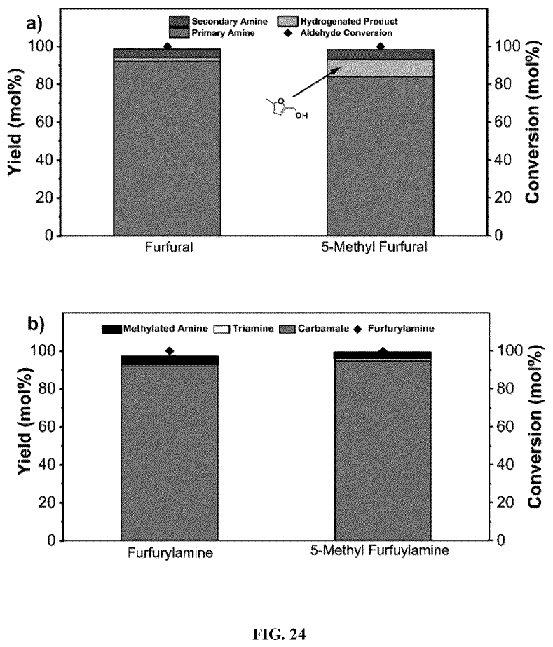

This synthesis pathway involved reductive amination and carbonylation steps. Methyl FC and FC were synthesized and isolated using a 2-step synthesis method shown in FIG. 21. The isolated yields for the carbamate are reported in Table 13 and no further tuning of reaction conditions was performed for either compound. The results of each step are shown in FIG. 24. FC was isolated as a dark brown oil, and Methyl FC was a golden-brown powder.

TABLE 13
Isolated Yields of the Carbamate^{a}

| | Methoxy carbonylation cumulative |
|---|---|
| Compound | isolated yield (mol %) |
| FC | 74.3 |
| Methyl FC | 72.6 |

^{a}= Reaction conditions of reductive amination: 10 mL liquid ammonia, 0.25 g furfural, 0.05 g Rh/Al₂O₃, 800 rpm stirring, 40 bar H₂, 100° C., 30 minutes. Reaction conditions of methoxy carbonylation: 80° C., 10 hours, 30 mmol DMC, 3 mmol furfurylamine, and 0.3 mmol La(OTf)₃.

HMFC and HMFC OMe Synthesis from Hydroxymethyl Furfural by Reductive Amination and Aminolysis of Chloroformate

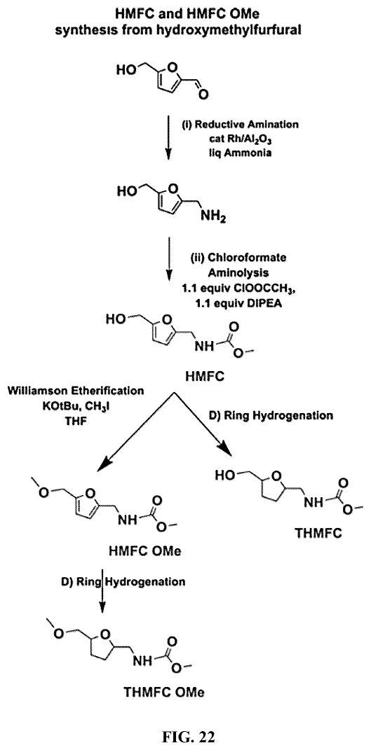

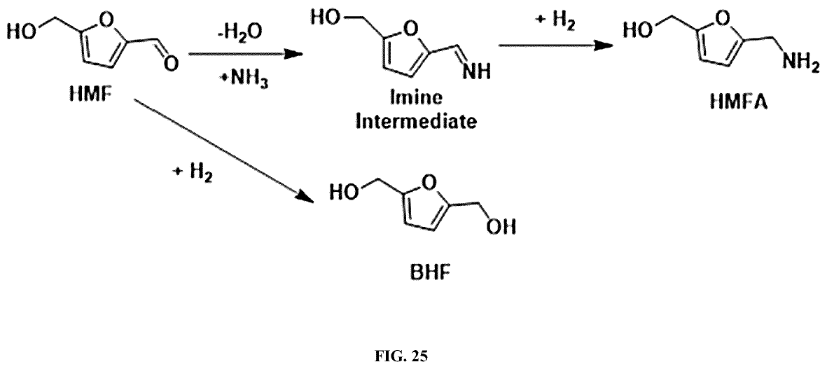

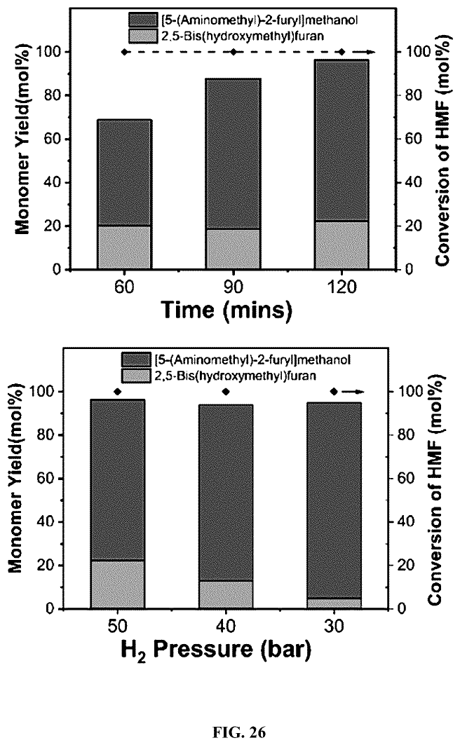

The synthesis involved 3 steps as shown in FIG. 22. Due to the potentially reactive hydroxyl group of HMF, initial reductive amination was performed under the established conditions of vanillylamine. The synthesis scheme is shown in FIG. 25. Further optimizations were needed to maximize the yield of 5-((aminomethyl)-2-furyl) methanol (HMFA). Initially, a 48% yield was obtained for the desired amine product, with the main byproduct being the diol, 2,5-bis(hydroxymethyl) furan (BHF) (20% yield), which formed from the aldehyde group hydrogenation. Reaction time and hydrogen pressure were tuned to increase the selectivity to the amine. Increasing the reaction time from 60 to 90 and 120 minutes increased the amine yield to 74% (FIG. 26). Notably, the carbon balance improved from 68% to 96% with increasing reaction time, indicating the presence of an imine intermediate. The hydrogen pressure optimization minimized the diol byproduct to 4%. The selectivity to the amine was maximized at a hydrogen pressure of 30 bar (91%). The high pH of the solution resulted in the ionization of HMFA, complicating its recovery from the aqueous phase. The hydroxyl group (pka 9.55) could be deprotonated in liquid ammonia (pH about 12), resulting in extraction difficulties of the amine from the aqueous phase. To maximize recovery, a procedure was adopted from Dunbabin et al (Dunbabin, A.; Subrizi, F.; Ward, J. M.; Sheppard, T. D.; Hailes, H. C. Furfurylamines from Biomass: Transaminase Catalysed Upgrading of Furfurals. Green Chem. 2017, 19 (2), 397-404) and the pH of the liquid ammonia was reduced to about 10 using 1 M HCl, and then extraction was performed following the procedures discussed above. No traces of the BHF product were observed in the isolated HMFA product.

Following the amine extraction, the carbamate was synthesized. While the previous method adhered to green principles, FC and Methyl FC were selectively produced using a 10-fold excess of DMC. Under similar conditions, the synthesis of HMFC was not selective. HMFA was insoluble in DMC and resulted in heavier products rather than the desired carbamate.

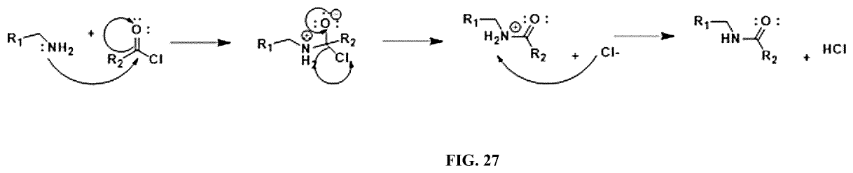

Given these complications, an alternate pathway was chosen to produce the HMFC-based carbamate effectively. Lautens et al previously reported a method for preparing carbamates via aminolysis of chloroformate in the presence of an aprotic base (Strategy, A. N.; Ring, I.; Compounds, O.; Lautens, M.; Kumanoviclb, S. Bicyclo[5.3.0]Decenes via Anionic Intramolecular Ring. 1995, 1954-1964). The mechanism, illustrated in FIG. 27, involves a nucleophilic amine attacking the carbonyl carbon, leading to the release of HCl. A stoichiometric amount of DIPEA was introduced to prevent unreacted amine from forming a salt with the released HCl. This approach capitalized on the amine's greater nucleophilicity than alcohol, maximizing selectivity to the desired carbamate. The shorter reaction time (30 minutes compared to 8 hours) and lower temperature (about 4° C.) were advantageous for this route.

As detailed in the methods section, HMFA and DIPEA (1.1 molar equivalents) were dissolved in dichloromethane, and the reaction was commenced by adding chloroformate (1.1 molar equivalents). Initial reactions produced slight effervescence upon adding chloroformate, which necessitated conducting subsequent tests in an ice bath maintained at about 4° C. Under the initial conditions, an 8% dimethyl carbonate side product yield was still observed. To eliminate this side reaction, a slight excess of the amine was used (1.1 molar equivalents), and the addition of the chloroformate (0.9 molar equivalents) was modified by dissolving it in 5 mL of dichloromethane and adding it dropwise. The pure carbamate was subsequently recovered using the solvent extraction method described in the experimental procedures. HMFC was isolated as a yellow powder. By modifying these established reactions and extraction based on the functional groups of the building blocks, high product yield and selectivity was achieved while further purification steps, such as column chromatography, which are time-consuming and require large amounts of solvents, were eliminated.

### Williamson Etherification of HMFC

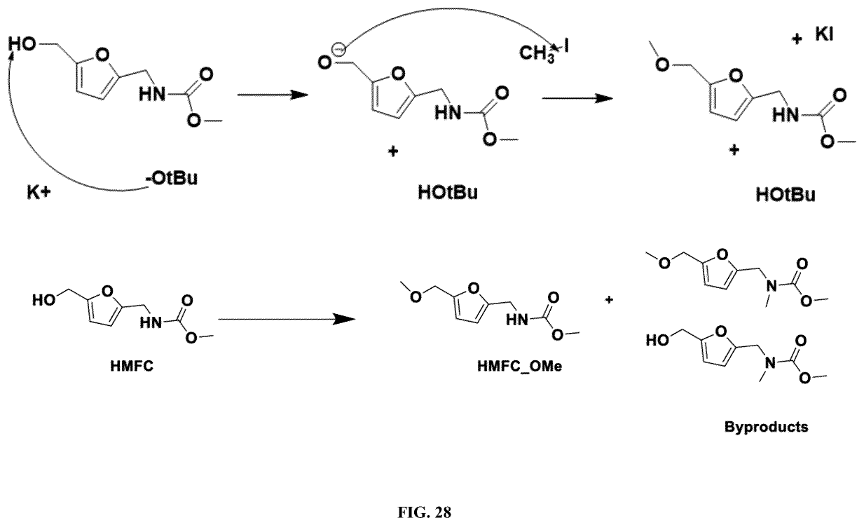

HMFC was converted into HMFC OMe using the Williamson ether synthesis method as shown in FIG. 28. This etherification required strong bases such as KOH or KOtBu first to deprotonate the alcohol to form an alkoxide ion, which performs a nucleophilic attack on an alkyl halide to yield an ether. KOtBu demonstrated 100% HMFC OMe conversion after 6 hours of reaction. A yield of 74% for HMFC OMe, with a 6% yield resulting from the alkylation of the NH group and 12% yield from alkylation of the OH and NH groups was observed. The selectivity favored HMFC OMe due to the higher basicity of the alcohol group. Due to the higher polarity and basicity of the side products, they were removed by washing the organic phase with a 2 M HCl solution. Pure HMFC OMe was obtained with a 52.9% isolated yield as a brown powder, 21% was lost due to the aqueous wash. The transformation to the ether group was particularly important for SAR as it indicates the impact of removing the hydrogen bond donor on HMFC.

### Synthesis of the Carboxylic Acid Carbamate: Amine Synthesis Via Azidation and Reduction, Aminolysis, and Base Hydrolysis of the Ester Carbamate

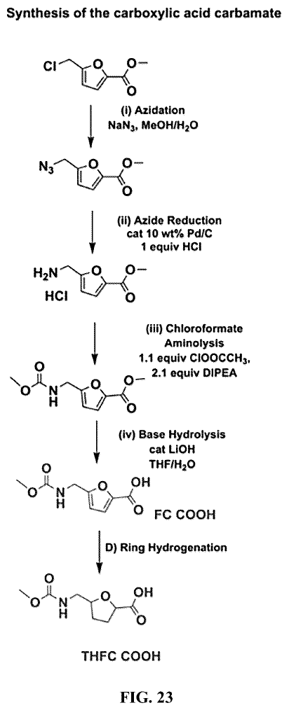

The carboxylic acid moiety was synthesized from methyl 5-(chloromethyl)-2-furoate (5 CMF Ester), derived from 5-chloromethyl furfural, a known biobased monomer. The synthesis method involves four steps, as shown in FIG. 23.

The amine synthesis began with the azidation of CMF Ester, following established literature procedures. Upon completion of the reaction, a 99% conversion of chloromethyl furoate and 96% selectivity to the azide were achieved. Additionally, an ether byproduct was identified, resulting from the nucleophilic attack of the methanol solvent.

The azide was reduced over 10 wt % Pd/C with a stoichiometric amount of HCl. The acid was introduced to precipitate the amine as a hydrochloric acid salt, 5-aminomethyl-furan-2-carboxylic methyl ester hydrochloride (5Amino Ester HCl). This step simplified the purification by separating the product from the unreacted chloromethyl furoate ester and the ether byproduct. After the reduction, the solvent evaporated, and the collected amine salt was washed repeatedly with diethyl ether to remove residual organic compounds. The isolated yield of the amine salt was 87%.

Aminolysis was conducted directly on the hydrochloride salt. The procedure for HMFC was modified to include an additional equivalent of DIPEA, which facilitated the release of the free amine from its initial salt form. The methyl ester ETMFC was obtained with an isolated yield of 92%, as determined by gravimetric analysis. ETMFC appeared as a yellow-white powder.

The carboxylic acid (FC COOH) was introduced through base-catalyzed hydrolysis of the ester-based carbamate. Acid hydrolysis was not selected due to its reversibility and side reactions of the basic carbamate group. Using lithium hydroxide (LiOH), base hydrolysis was performed in a THF/water mixture at 50° C. for 1 hour, following established literature. This protocol was applied to the furoate carbamate, and the pure carboxylic acid carbamate was obtained with an isolated yield of 88% (cumulative 70%).

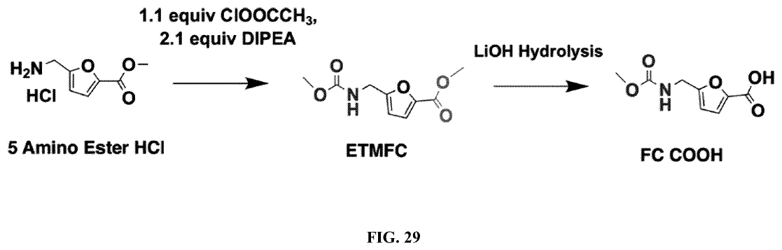

The selective hydrolysis of the ester group over carbamate on the —NH group is shown in FIG. 29. This conversion was confirmed by H¹-NMR. The larger shielding effect of the electron lone pair on the NH group compared to the lone pair from the furan ring caused the carbamate ether to appear further upfield, referencing the 3H peak at 3.55 ppm, compared to the ether group of the ester carbamate (3.8 ppm). The disappearance of the downfield ether group (3.8 ppm) confirmed selective hydrolysis of the ester.

### Ring Hydrogenation

Rather than start from tetrahydrofuran building blocks, ring hydrogenation was performed on the furan carbamates. Pd catalysts are known for their high activity in reducing furan rings in aqueous and organic solvents. While water is an excellent solvent for reduction, isopropanol solvent was used to simplify the recovery of the hydrogenated products. Initial optimizations with FC resulted in complete conversion to THFC with a selectivity of 99% after 3 hours (Table 14).

TABLE 14
Time-dependent Studies of FC Reduction over 5 wt % Pd/C.

| Time (min) | Conversion of FC | Yield of THFC |
|---|---|---|
| 30 | 20.1 | 18.3 |
| 60 | 42.1 | 37.7 |
| 180 | 100 | 98.8 |

Reaction Conditions: 0.25 g of carbamate, 0.02 g 5 wt % Pd/C, 3 hours, 80° C., 40 bar H₂, 15 mL Isopropyl alcohol.

For HMFC and HMFC OMe ring hydrogenation, Methyl THFC was observed resulting from C—C cracking. The yields were minimal (<4%) for HMFC; however, in some replicates, the purity of THMFC OMe dropped to 89% due to the Methyl THFC byproduct, identified through GCMS analysis. The averages across 3 experiments are reported in Table 15.

TABLE 15
Results of Ring Hydrogenation Reactions Conducted over 5 wt %
Pd/Cª

| | | | Ring | |
|---|---|---|---|---|
| | | | Hydrogenat- | By- |
| | Conversion | Ring Hydrogenated | ed Product | product |
| Reactant | (%) | Product Structure | Yield (%) | Yield (%)* |
| FC | 100 | 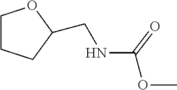 | 98.8 | 0 |
| HMFC | 100 | 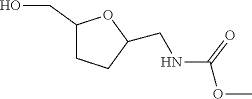 | 94.4 | 4.5 |
| HMFC OMe | 100 | 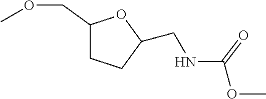 | 92.9 | 5.7 |
| Methyl FC | 100 | 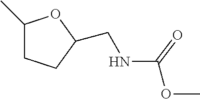 | 97.7 | 0 |
| FC COOH* | 97.4 | 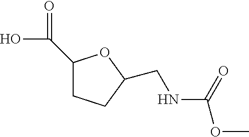 | 91.2 | 4.1 |

^{a}Reaction Conditions: 0.25 g of carbamate, 0.02 g 5 wt % Pd/C, 3 hours, 80° C., 40
bar H₂, 15 mL of Isopropyl alcohol.
*100° C., time 4 hours.
^{#}Byproducts were primarily methyl THFC.

The formation of diastereomers was observed from the reduction of Methyl FC, HMFC, and HMFC OMe. The ratio of isomers in each case was 7.1:1 for HMFC, 3.28:1 for HMFC OMe and 2.8:1 for Methyl FC, calculated based on the GCMS areas of each peak. The favored molecule was likely the cis isomer, as syn addition of H₂ is generally favored over noble metal catalysts. The reduction conditions were then tuned for FC COOH due to the delocalizing effect of the carboxylic acid substituent; its electron-withdrawing nature reduces the nucleophilicity of the furan ring, making it less susceptible to hydrogenation. The initial conditions resulted in 7% acid conversion, but the reaction proceeded smoothly at 100° C., resulting in 97% conversion of FC COOH. GCMS analysis revealed the formation THFC in the product mixture at 4% yield along with traces of butylated hydroxytoluene (THF stabilizer).

### Insecticidal Activity

Bioassays were performed to assess the molecules' insecticidal potency against the lesser mealworm beetle, Alphitobius diaperinus, using acetone as the control and an industrial standard carbofuran. For comparison, an ester group carbamate was evaluated alongside the carboxylic acid-functionalized FC COOH, and similarly for HMFC and HMFC OMe. FC and THMFC OMe exhibited distinctly higher toxicity among the assessed carbamates than the other compounds. Conversely HMFC and FC COOH showed no toxicity both on a gram and mole basis, likely due to the susceptibility of the OH and COOH groups to phase II metabolism. Additionally, the higher polarity of the compounds would likely limit their diffusion across cell membranes composed of a hydrophobic phospholipid bilayer. The remaining compounds had LC₅₀ values that broadly overlapped, ranging from 0.58 to 1.71 mg/cm². THFC COOH and THFC OMe contained minor impurities (about 4% THFC and about 5% Methyl THFC, respectively). Methyl THFC had a higher LC₅₀ (0.66 mg/cm²) than THMFC OMe (0.2 mg/cm²) and therefore would not contribute to its performance. Regarding THFC COOH, its highest test dosage was 6.09 mg/cm², the relative amount of THFC would be about 0.2 mg/cm², 4 times lower than its lower bound toxicity 0.94 mg/cm². THFC's LC₅₀ value, combined with low relative concentrations, place it below toxic thresholds and unlikely to contribute to observed activity.

The LC₅₀ estimates from probit analysis, presented in Table 16, provided insights into the structure-activity relationships governing potency.

TABLE 16
Probit Analysis Results for Adult Alphitobius Diaperinus

| | | LC₅₀ | | |
|---|---|---|---|---|
| Compound IUPAC Name | Code | (mg/cm²) | | LC₅₀(mmol/cm²) |
| Methyl N-[(furan-2-yl)methyl]carbanate | FC | 0.22 | [0.16, 0.34] | 1.42E−03 |
| Methyl N-[(oxolan-2- | THFC | 1.28 | [0.94, 1.53] | 8.05E−03 |
| yl)methyl]carbamate | | | | |
| Methyl N-{[5-(hydroxymethyl)furan-2- | HMFC | N/A | | NA |
| yl]methyl}carbanate | | | | |
| Methyl N-{[5-(hydroxymethyl)oxolan-2- | THMFC | 1.71 | [1.26, 2.78] | 9.05E−03 |
| yl]methyl}carbamate | | | | |
| Methyl N-{[5-(methoxymethyl)furan-2- | HMFC OMe | 1.39 | [1.08, 1.73] | 6.98E−03 |
| yl]methyl}carbamate | | | | |
| Methyl N-{[5-(methoxymethyl)oxolan- | THMFC OMe | 0.20 | [0.09, 0.36] | 9.85E−04 |
| 2-yl]methyl}carbamate | | | | |
| 5- | FC COOH | N/A | | N/A |
| {[(methoxycarbonyl)amino]methyl}furan-2- | | | | |
| carboxylic acid | | | | |
| methyl 5- | ETMFC | 1.87 | [0.55, 5.68] | 8.77E−03 |
| {[(methoxycarbonyl)amino]methyl}furan-2- | | | | |
| carboxylate | | | | |
| 5- | THFC COOH | 2.04 | [0.28, 3.18] | 10.04E−03 |
| {[(methoxycarbonyl)amino]methyl}oxolane- | | | | |
| 2-carboxylic acid | | | | |
| methyl N-[(5-methylfuran-2- | Methyl FC | 0.58 | [0.03, 0.89] | 3.43E−03 |
| yl)methyl]carbamate | | | | |
| methyl N-[(5-methyloxolan-2- | Methyl THFC | 0.66 | [0.58, 0.77] | 3.82E−03 |
| yl)methyl]carbamate | | | | |
| 2,3-dihydro-2,2-dimethylbenzofuran-7- | Carbofuran | 0.25 | [0.11, 0.56] | 1.13E−03 |
| yl methylcarbamate | | | | |

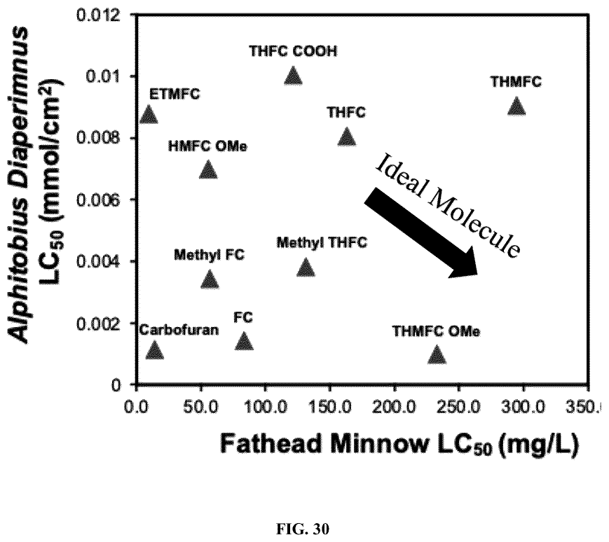

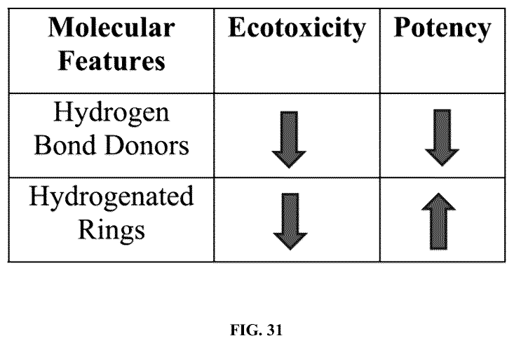

A general trend observed in the bioassay was that furan compounds linked with hydrogen donor groups, such as hydroxyl or carboxylic acid, showed no toxicity to Alphitobius diaperinus, even at high concentrations, with mortality rates similar to the control. In contrast, significant mortality shifts were observed when these hydrogen bonds were replaced by ether linkages (e.g., hydroxyl to ether and acid to ester). Additionally, hydrogenating the furan rings with oxygen functional groups resulted in a marked increase in toxicity, a trend seen across ether, hydroxyl, and carboxylic acid-substituted furans. To assess comparative potency and ecotoxicity, the LC₅₀ values of the compounds were plotted against their toxicity toward the fathead minnow, shown in FIG. 30. All the top ten filtered compounds displayed superior ecotoxicity to carbofuran, while Methyl FC, Methyl THFC, FC and THMFC OMe displayed comparable performance to the lesser mealworm beetle. THMFC OMe was identified as a lead molecule due to its superior ecotoxicity, which is attributed to its hydrogenated furan ring. FIG. 31 outlines the key takeaways from this analysis.

### Honeybee Toxicity Evaluations

The quantification of Honey bee adult acute contact was conducted against European honeybee (Apis mellifera), as a standard measure of safety for pollinators. The dosing solutions were prepared by measuring appropriate amounts of material for an 800 mg/mL of test substance in acetone. Subsequent dosing solutions were prepared by serial dilution from the highest test level in acetone. The negative control dosing solution consisted of water only, while the carrier control group was treated with acetone. All bees received a 2 μL dosing volume. When applied at 2 μL, the 800 mg/ml solution would result in a 1.6 mg/bee dose. A stock solution of the positive control substance was prepared by dissolving 0.0015 g of dimethoate in 10 mL of acetone. Dosing solutions for the positive control were prepared by proportional volumetric dilution (3.33 and 5 mL each diluted to 10 mL) with acetone. For dosing, bees were immobilized using CO₂ gas. The treatment was applied using a pipette, to the dorsal side of the bee's thorax (between the wings). Bees were then placed back into observation cages and stored inside a growth chamber for mortality observations over the next two days.

Bees for the study were housed in ventilated stainless steel exposure cages with glass windows, measuring 8.5 by 4.5 by 6.5 cm. A plastic, 10 ml syringe containing 50% (w/v) aqueous sucrose solution was inserted through the lid of the chamber, allowing the bees to feed ad libitum throughout the test period. During the test, cages were maintained in an environmental chamber. The bees were maintained in continuous darkness during the test except during periods of dosing and observations. Temperature and humidity were measured continuously during the test, with a temperature range of 79 to 88° F. and relative humidity ranging from 43 to 63%.

As shown in Table 17, promisingly, the 24-hr LD50 of our compound was 126 μg/bee (and 48-hr LD50=75 μg/bee), which is orders of magnitude less toxic than carbamate or neonicotinoid insecticides, and on par with newer diamide insecticides that are considered generally safe for honeybees and other pollinators (see e.g., Hardstone MC, Scott JG. 2010. “Is Apis mellifera more sensitive to insecticides than other insects?” Pest Manag. Sci. 66 (11): 1171-1180; Li W, Zan Y, Wu T, et al. 2024. “Impact of chlorantraniliprole on honey bees: Differential sensitivity and biological responses in Apis mellifera and Apis cerana.” Sci. Total Environ.; and Ma A, Hm N, Hm A, et al. “Acute Toxicity of Selected Insecticides and Their Safety to Honey Bee (Apis mellifera L.)” Workers Under Laboratory Conditions).

TABLE 17
Honeybee adult acute contact test results for FC

| Treatment | | | | | | | | | |
|---|---|---|---|---|---|---|---|---|---|
| Group | | Mortality | | | Total | | Average | | Average |
| (ug a.i/ | | | Day | Day | number | 24-hr % | 24-hr % | 48-hr % | 48-hr % |
| bee) | Rep | 4-hour | 1 | 2 | of Bees | Mortality | Mortality | Mortality | Mortality |
| Negative | 1 | 0 | 0 | 0 | 10 | 0 | 0 | 0 | 0 |
| Control | 2 | 0 | 0 | 0 | 10 | 0 | | 0 | |
| | 3 | 0 | 0 | 0 | 10 | 0 | | 0 | |
| Carrier | 1 | 0 | 0 | 0 | 10 | 0 | 0 | 0 | 0 |
| Control | 2 | 0 | 0 | 0 | 10 | 0 | | 0 | |
| | 3 | 0 | 0 | 0 | 10 | 0 | | 0 | |
| | 1 | 0 | 0 | 0 | 10 | 0 | 0 | 0 | 0 |
| 0.0016 | 2 | 0 | 0 | 0 | 10 | 0 | | 0 | |
| | 3 | 0 | 0 | 0 | 10 | 0 | | 0 | |
| | 1 | 0 | 0 | 0 | 10 | 0 | 3 | 0 | 3 |
| 0.16 | 2 | 0 | 1 | 1 | 10 | 10 | | 10 | |
| | 3 | 0 | 0 | 0 | 10 | 0 | | 0 | |
| | 1 | 0 | 0 | 0 | 10 | 0 | 0 | 0 | 0 |
| 16 | 2 | 0 | 0 | 0 | 10 | 0 | | 0 | |
| | 3 | 0 | 0 | 0 | 10 | 0 | | 0 | |
| | 1 | 0 | 2 | 2 | 10 | 20 | 10 | 20 | 10 |
| 40 | 2 | 0 | 1 | 1 | 10 | 10 | | 10 | |
| | 3 | 0 | 0 | 0 | 10 | 0 | | 0 | |
| | 1 | 0 | 6 | 8 | 10 | 60 | 43 | 80 | 57 |
| 80 | 2 | 0 | 4 | 6 | 10 | 40 | | 60 | |
| | 3 | 0 | 3 | 3 | 10 | 30 | | 30 | |
| | 1 | 0 | 7 | 10 | 10 | 70 | 53 | 100 | 90 |
| 160 | 2 | 0 | 2 | 7 | 10 | 20 | | 70 | |
| | 3 | 0 | 7 | 10 | 10 | 70 | | 100 | |
| | 1 | 0 | 10 | 10 | 10 | 100 | 100 | 100 | 100 |
| 1600 | 2 | 0 | 10 | 10 | 10 | 100 | | 100 | |
| | 3 | 0 | 10 | 10 | 10 | 100 | | 100 | |
| | 1 | 0 | 1 | 2 | 10 | 10 | 10 | 20 | 13 |
| Positive | 2 | 0 | 2 | 2 | 10 | 20 | | 20 | |
| Control | | | | | | | | | |
| (0.05) | 3 | 0 | 0 | 0 | 10 | 0 | | 0 | |
| | 1 | 0 | 6 | 9 | 10 | 60 | 73 | 90 | 83 |
| Positive | 2 | 0 | 8 | 8 | 10 | 80 | | 80 | |
| Control | | | | | | | | | |
| (0.10) | 3 | 1 | 8 | 8 | 10 | 80 | | 80 | |
| | 1 | 4 | 10 | 10 | 10 | 100 | 100 | 100 | 100 |
| Positive | 2 | 0 | 10 | 10 | 10 | 100 | | 100 | |
| Control | | | | | | | | | |
| (0.30) | 3 | 4 | 10 | 10 | 10 | 100 | | 100 | |

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

1. A process for preparing a bio-based carbamate functionalized insecticide comprising:
performing a reductive amination reaction on an aldehyde to produce an amine product; and
subjecting the amine product to a carbonylation reaction to produce the bio-based carbamate insecticide.

2. The process of claim 1, wherein the bio-based carbamate functionalized insecticide is either a compound of Formula (I): 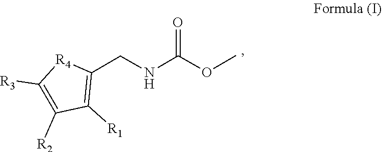
wherein:
R₁ is H,
R₂ is H,
R₃ is H, —CH₃ or —CH₂Cl, —CH₂Br, —CH₂I, —CH₂OH, —CH₂OCH₃, —COOH and
R₄ is O, S or NH;
a compound of Formula (II): 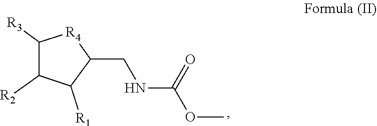
wherein:
R₁ is H,
R₂ is H,
R₃ is H, —CH₃ or —CH₂Cl, —CH₂Br, —CH₂I, —CH₂OH, —CH₂OCH₃, —COOH and
R₄ is O, S or NH;
a compound of Formula (III) 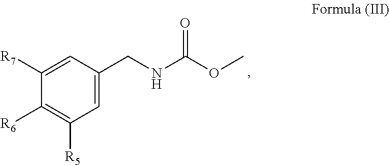
wherein:
R₅ is —OCH₃, —OH or H,
R₆ is —OCH₃, —OH or H, and
R₇ is —OCH₃, OH or H; or
a compound with a structure of:

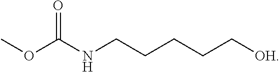

3. The process of claim 1, wherein the aldehyde is selected from:

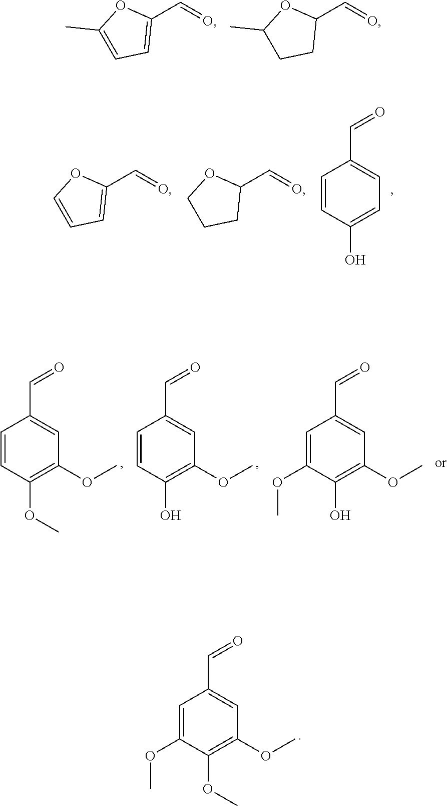

4. The process of claim 1, wherein the performing of the reductive amination reaction comprises reacting the aldehyde with ammonia under a H₂ pressure ranging from 30 bar to 60 bar.

5. The process of claim 1, wherein the reductive amination reaction occurs for about 30 minutes to about 120 minutes.

6. The process of claim 1, wherein the reductive amination reaction is performed at a temperature ranging from 80° C. to 100° C.

7. The process of claim 1, wherein the reductive amination reaction is performed in a presence of a first catalyst, that catalyst being selected from Rh/Al₂O₃, Pd/C, Ru/C, Ru/ZrO₂, Raney Nickel, Rh/C or Pt/C.

8. The process of claim 1, wherein the subjecting of the amine product to the carbonylation reaction comprises reacting the amine product in a presence of a metal triflate catalyst in dimethyl carbonate.

9. The process of claim 8, wherein the metal triflate catalyst is ytterbium triflate, lanthanum triflate, scandium triflate or zinc triflate.

10. The process of claim 8, wherein dimethyl carbonate is present in the carbonylation reaction in a concentration ranging from 20 mmol to 60 mmol.

11. The process of claim 1, wherein the carbonylation reaction occurs for about 8 to about 16 hours.

12. The process of claim 1, wherein the carbonylation reaction comprises reacting the amine product with dimethyl carbonate (DMC) and a catalyst in a DMC/amine/catalyst ratio of 10:1:0.1.

13. The process of claim 1, wherein the reductive amination reaction produces the amine product in a yield ranging from about 85% to about 100%.

14. The process of claim 1, wherein the carbonylation reaction produces the bio-based carbamate functionalized insecticide in a yield ranging from 60% to about 95%.

15. The process of claim 1, the process comprising:
isolating the aldehyde from a renewable resource containing lignosulfonates, kraft and/or alkali lignin.

16. The process of claim 15, wherein the isolating of the aldehyde from the renewable resource comprises:
depolymerizing the lignosulfonates to produce a reaction mixture containing the aldehydes, and
precipitating the aldehydes out of the reaction mixture.

17. The process of claim 15, wherein the renewable resource is cellulosic biomass.

18. An insecticidal composition comprising a bio-based carbamate functionalized insecticide produced from the process of claim 1.

19. The insecticidal composition of claim 17, wherein the composition has an LC₅₀ against adult Alphitobius diaperinus of at least 6,660,622 ng/cm³.

20. A process for preparing a bio-based insecticide with active carbamate functional group insecticide comprising:
performing a reductive amination reaction on an aldehyde to produce an amine product; and
subjecting the amine product to a carbonylation reaction to produce the bio-based carbamate insecticide,
wherein the aldehyde is selected from the group consisting of:

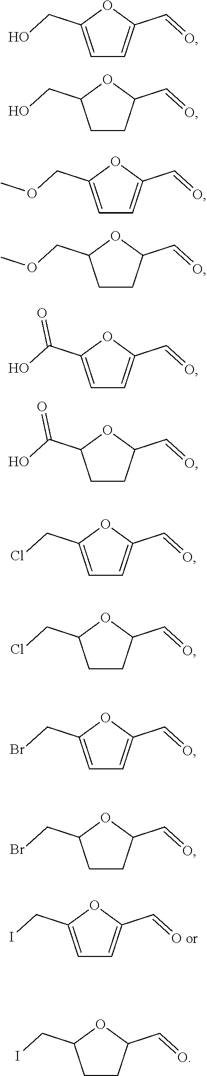

21. A process for preparing a bio-based carbamate functionalized insecticide comprising:
performing an azidation reaction on a methyl-ester containing an alkyl halogen group to produce an azide intermediate;
subjecting the azide intermediate to an azide reduction reaction to produce an amine intermediate;
performing an aminolysis reaction on the amine intermediate to produce an ester carbamate intermediate;
subjecting the ester carbamate intermediate to a hydrolysis reaction to produce the bio-based carbamate functionalized insecticide; and
optionally performing a ring hydrogenation on the bio-based carbamate functionalized insecticide.

22. The process of claim 21, wherein the azidation reaction is performed on an ester having a structure of Formula (VI): 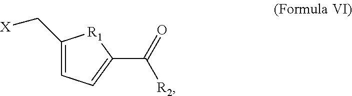
wherein:
R₁ is O, S or NH,
R₂ is OR₃, wherein R₃ is a C₁-C₆ alkyl chain, and
X is a halogen.

23. The process of claim 21, wherein the azide intermediate has a structure of Formula (VII): 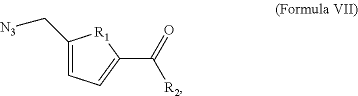
wherein:
R₁ is O, S or NH, and
R₂ is OR₃, wherein R₃ is a C₁-C₆alkyl chain.

24. The process of claim 21, wherein the amine intermediate has a structure of Formula (VIII): 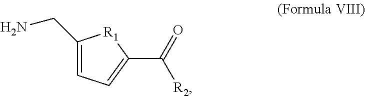
wherein:
R₁ is O, S or NH, and
R₂ is OR₃, wherein R₃ is a C₁-C₆alkyl chain.

25. The process of claim 21, wherein the ester carbamate intermediate has a structure of Formula IX: 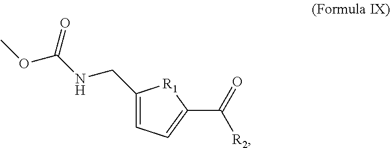
wherein:
R₁ is O, S or NH, and
R₂ is OR₃, wherein R₃ is a C₁-C₆ alkyl chain.

26. The process of claim 21, wherein the bio-based carbamate functionalized insecticide produced from the hydrolysis reaction has a structure of Formula X: 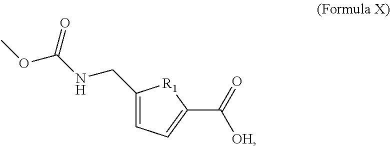
wherein:
R₁ is O, S or NH.

27. The process of claim 21, wherein the process comprises performing the ring hydrogenation on the bio-based carbamate functionalized insecticide to produce a bio-based carbamate functionalized insecticide having a structure of Formula XI: 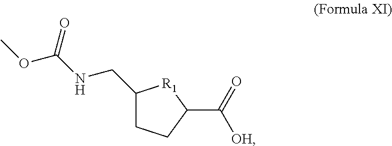
wherein:
R₁ is O, S or NH.
